SEQUENCE LISTING (1 sequence)

SEQ ID NO: 1 - protein, 223 aa, Homo sapiens
MQWAVGRRWAWAALLLAVAAVLTQVVWLWLGTQSFVFQREEIAQLARQYAGLDHELAFSRLIVELRRLHPGHVLPDEELQWVFVNAGGWMGAMCLLHASLSEYVLLFGTALGSRGHSGRYWAEISDTIISGTFHQWREGTTKSEVFYPGETVVHGPGEATAVEWGPNTWMVEYGRGVIPSTLAFALADTVFSTQDFLTLFYTLRSYARGLRLELTTYLFGQDP

(19) United States
(12) Patent Application Publication
(10) Pub. No.: US 2024/0307325 A1
(43) Pub. Date: Sep. 19, 2024

(54) COMPOUNDS, COMPOSITIONS, AND METHODS FOR TREATING OR AMELIORATING NONALCOHOLIC FATTY LIVER DISEASE AND RELATED DISEASES OR DISORDERS

(71) Applicant: Thomas Jefferson University, Philadelphia, PA (US)

(72) Inventor: Felix J. Kim, Philadelphia, PA (US)

(21) Appl. No.: 18/272,486

(22) PCT Filed: Jan. 14, 2022
(86) PCT No.: PCT/US22/12516

§ 371 Date: Jul. 14, 2023

Related U.S. Application Data
(60) Provisional application No. 63/137,915, filed on Jan. 15, 2021.

(51) Int. Cl. A61K 31/155; A61K 31/7105; A61K 31/713; A61P 1/16; A61P 29/00; C12N 15/86; G01N 33/68

(52) U.S. Cl. CPC A61K 31/155; A61K 31/7105; A61K 31/713; A61P 1/16; A61P 29/00; C12N 15/86; G01N 33/6893; C12N 2740/15043; G01N 2333/705; G01N 2800/085; G01N 2800/50

(57) ABSTRACT

The present disclosure relates to the finding that certain compounds that modulate the activity(ies) of Sigma receptors can be used to treating or ameliorate nonalcoholic fatty liver disease and related diseases or disorders. In certain embodiments, the Sigma receptor is a Sigma-I receptor (also known as Sigma I).

### CROSS-REFERENCE TO RELATED APPLICATIONS

The present application claims priority under 35 U.S.C. § 119(e) to U.S. Provisional Patent Application No. 63/137,915, filed Jan. 15, 2021, which is incorporated herein by reference in its entirety.

### BACKGROUND

Nonalcoholic fatty liver disease (NAFLD) describes a continuum of hepatic abnormalities starting with hepatic steatosis (fatty liver). In a subset of patients, NAFLD will progress to nonalcoholic steatohepatitis (NASH). NASH is defined histologically by hepatic steatosis with lobular inflammation, hepatocyte injury (ballooning), with or without fibrosis. NASH can then progress/lead to cirrhosis and hepatocellular carcinoma (HCC).

NAFLD is a steadily increasing global cause of chronic liver disease, along with worldwide increase of obesity and metabolic syndrome. In the US, approximately 24% of the population is estimated to have NAFLD, with approximately a third of these people having NASH. NAFLD is associated with a spectrum of comorbidities including metabolic syndrome, diabetes, and cardiovascular disease (CVD). NAFLD is the fastest growing cause of HCC among liver diseases, and among the leading diseases requiring liver transplantation. Although considerable progress has been made in elucidating NAFLD pathogenesis as well as in the identification of therapeutic targets and the discovery and development of new drugs, there remain important unanswered questions in the field and unmet clinical needs for this patient population.

Factors involved in NAFLD progression are not well defined. NAFLD has a highly variable natural history reflected by different rates of progression among individuals and has heterogeneous clinical manifestations. Underlying these differences are the convergence of various environmental and social/behavioral influences, as well as metabolic comorbidity, gut microbiome, genetic, gender, and age associated/related risk factors. Thus, the mechanisms driving NAFLD and the clinical manifestation are heterogeneous. The factors involved in natural history of the disease and its progression are not well defined. Several molecular pathways have been described to contribute to the development of NASH, however, the pathogenic drivers of NAFLD and the molecular mechanisms leading to and driving disease are not well defined but are likely to differ among patients and are likely to be multi-focal.

Methods to identify the approximately 20% of NAFLD patients whose NASH rapidly progresses to advanced fibrosis remains elusive, and the factors underlying this rapid progression remain unclear. There is a pressing need for predictive biomarkers of disease risk and pharmacodynamic biomarkers of drug response. However, it remains a challenge to identify people at risk for NASH due to inadequate understanding of the natural history of NAFLD and of the key pathogenic drivers and enablers/facilitators of the disease.

Currently treatment options for NALFD remain limited, and there are no FDA-approved drugs to treat patients affected by this rapidly growing public health concern. There is a pressing need for new therapeutic agents and treatment strategies as well as biomarkers of disease risk and drug response. Several agents are in clinical trials, including four distinct targeted agents in at least six phase 3 trials to treat NASH with fibrosis. Of these, two recently completed Phase III clinical trials of ASK-1 inhibitor selonsertib failed to meet pre-specified week 48 primary endpoints of ≥1-stage histologic improvement in fibrosis without worsening of NASH.

This highlights the need for biomarker guided therapeutic trials. Therapeutic agents and diagnostic technologies are crucial to more accurately predict disease progression and develop more effective treatments and more effectively design clinical trials. Clinical trial design and strategies are rapidly evolving with a growing list of drug targets and better understanding of the factors that underlie the heterogeneity of NAFLD.

There is a need in the art to identify novel compounds, compositions, methods, and biomarkers that allow for treating, preventing, and/or monitoring NAFLD and related diseases. The present disclosure addresses this unmet need.

### SUMMARY

In certain aspects, the instant specification is directed to the following non-limiting embodiments:

Embodiment 1: A method of treating, ameliorating, and/or preventing accumulation of lipid droplets (LDs) in a cell, the method comprising contacting the cell with an agent that reduces and/or prevents SIGMAR1 transcription, and/or reduces and/or inhibits Sigma1 activity, localization, and/or expression, in the cell.

Embodiment 2: A method of treating, ameliorating, and/or preventing hepatic steatosis (fatty liver) and/or inflammation associated with steatohepatitis in a subject, the method comprising administering to the subject an effective amount of an agent that reduces and/or prevents SIGMAR1 transcription, and/or reduces and/or inhibits Sigma1 activity and/or expression, in the subject.

Embodiment 3: A method of treating, ameliorating, and/or preventing nonalcoholic fatty liver disease (NAFLD) in a subject, the method comprising administering to the subject an effective amount of an agent that reduces and/or prevents SIGMAR1 transcription, and/or reduces and/or inhibits Sigma1 activity and/or expression, in the subject.

Embodiment 4: A method of treating, ameliorating, and/or preventing nonalcoholic steatohepatitis (NASH) in a subject, the method comprising administering to the subject an effective amount of an agent that reduces and/or prevents SIGMAR1 transcription, and/or reduces and/or inhibits Sigma1 activity and/or expression, in the subject.

Embodiment 5: A method of reversing, slowing, or preventing progression of NAFLD to NASH in a subject suffering from NAFLD, the method comprising administering to the subject an effective amount of an agent that reduces and/or prevents SIGMAR1 transcription, and/or reduces and/or inhibits Sigma1 activity and/or expression, in the subject.

Embodiment 6: A method of reversing, slowing, or preventing progression of NASH to hepatocellular carcinoma (HCC) in a subject suffering from NASH, the method comprising administering to the subject an effective amount of an agent that modulates SIGMAR1 transcription, and/or modulates Sigma1 activity and/or expression, in the subject.

Embodiment 7: A method of determining and/or evaluating risk that a subject suffering from NAFLD will progress to NASH, the method comprising measuring levels of SIGMAR1 transcription and/or Sigma1 activity/expression in the subject, and comparing the measured levels to those levels of SIGMAR1 transcription and/or Sigma1 activity/expression in a control sample.

Embodiment 8: The method of embodiment 7, wherein the subject at risk of progressing to NASH is counseled to receive an effective amount of an agent that reduces and/or prevents SIGMAR1 transcription, and/or reduces and/or inhibits Sigma1 activity and/or expression.

Embodiment 9: The method of embodiment 7, wherein the subject at risk of progressing to NASH is administered an effective amount of an agent that reduces and/or prevents SIGMAR1 transcription, and/or reduces and/or inhibits Sigma1 activity and/or expression.

Embodiment 10: A method of determining and/or evaluating risk that a subject suffering from NASH will progress to HCC, the method comprising measuring levels of SIGMAR1 transcription and/or Sigma1 activity/expression in the subject, and comparing the measured levels to those levels of SIGMAR1 transcription and/or Sigma1 activity/expression in a control sample.

Embodiment 11: The method of embodiment 10, wherein the subject at risk of progressing to HCC is counseled to receive an effective amount of an agent that modulates SIGMAR1 transcription, and/or modulates Sigma1 activity and/or expression.

Embodiment 12: The method of embodiment 10, wherein the subject at risk of progressing to HCC is counseled to receive an effective amount of an agent that modulates SIGMAR1 transcription, and/or modulates Sigma1 activity and/or expression.

Embodiment 13: The method of any of embodiments 7-12, wherein the control sample is from at least of the following: a subject suffering from NAFDL; a subject not suffering from NAFDL; a subject suffering from NASH; a subject not suffering from NASH; a subject suffering from HCC; a subject not suffering from HCC; a subject suffering from NAFLD who has not progressed to NASH and/or is known not to be at risk of progressing to NASH; a subject suffering from NAFLD who has progressed to NASH and/or is known to be at risk of progressing to NASH; a subject suffering from NASH who has not progressed to HCC and/or is known not to be at risk of progressing to HCC; a subject suffering from NASH who has progressed to HCC and/or is known to be at risk of progressing to HCC.

Embodiment 14: The method of any of embodiments 2-6, wherein the administering reverses, prevents, and/or ameliorates at least one of the following: steatosis, inflammation, hepatocyte ballooning, fibrosis, hepatic stellate cell (HSC) activation, serum liver enzyme increase, insulin resistance/glucose tolerance, and HCC lesion formation.

Embodiment 15: The method of any of embodiments 1-6, 8-9, and 11-14, wherein the agent comprises a CRISPR system against SIGMAR1 and/or a nucleic acid against SIGMAR1 selected from a siRNA, a shRNA, a microRNA, or antisense polynucleotide.

Embodiment 16: The method of embodiment 15, wherein the subject is administered a viral vector expressing the nucleic acid.

Embodiment 17: The method of embodiment 16, wherein the nucleic acid is expressed in the liver of the subject.

Embodiment 18: The method of any of embodiments 1-6, 8-9, and 11-14, wherein the agent is:

a compound of Formula (I):

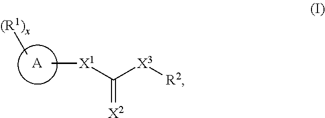

wherein:
ring A is a monocyclic or bicyclic aryl or a monocyclic or bicyclic heteroaryl ring, and wherein the aryl or heteroaryl ring is optionally substituted with 0-4 R¹ groups;
each occurrence of R¹ is independently selected from the group consisting of —C₁-C₆ alkyl, —C₁-C₆ fluoroalkyl, —C₁-C₆ heteroalkyl, F, Cl, Br, I, —CN, —NO₂, —OR³, —SR³, —S(═O)R³, —S(═O)₂R³, —NHS(═O)₂R³, —C(═O)R³, —OC(═O)R³, —CO₂R³, —OCO₂R³, —CH(R³)₂, —N(R³)₂, —C(═O)N(R³)₂, —OC(═O)N(R³)₂, —NHC(═O)NH(R³), —NHC(═O)R³, —NHC(═O)OR³, —C(OH)(R³)₂, and —C(NH₂)(R³)₂;
each occurrence of R² is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl or cycloalkyl group is optionally substituted with 0-5 R¹ groups, or X³ and R² combine to form a (C₃-C₇)heterocycloalkyl group, optionally substituted with 0-2 R¹ groups;
each occurrence of R³ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted with 0-5 R¹ groups;
X¹ is —CH₂—, —S—, —O— or —(NR₂)—;
X² is ═CH₂, ═S, ═O or ═NR₂; and
X³ is —S—, —O—, or —NR₂—; and
   a compound of Formula (II): R^{A}—R^{B} (II), wherein;
R^{A} is selected from the group consisting of

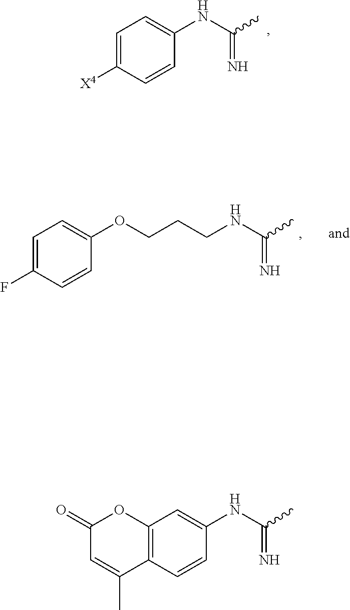

X⁴ is selected from the group consisting of methoxy, F, Cl, Br, and I; and
R^{B} is selected from the group consisting of:

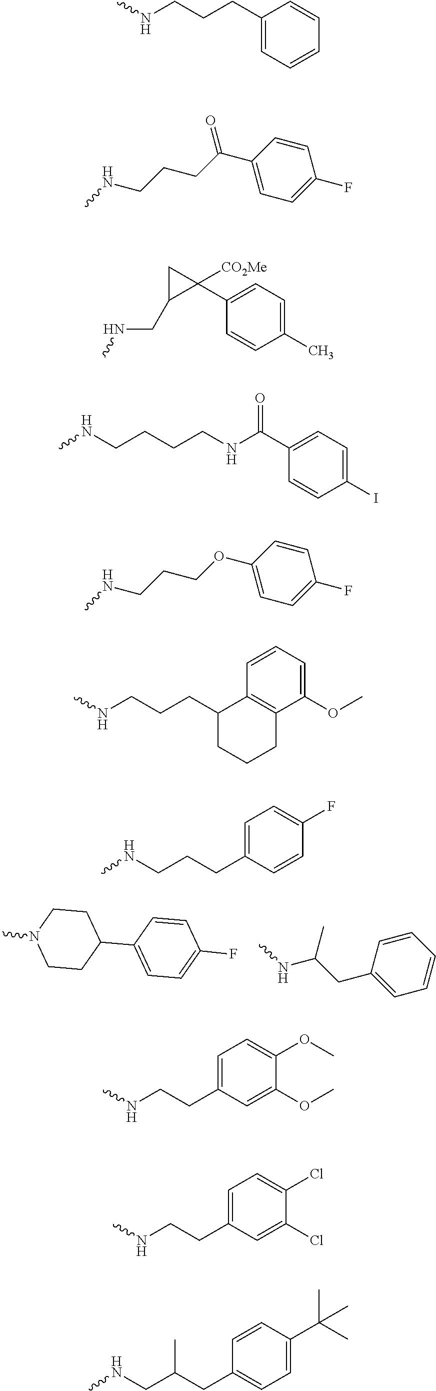

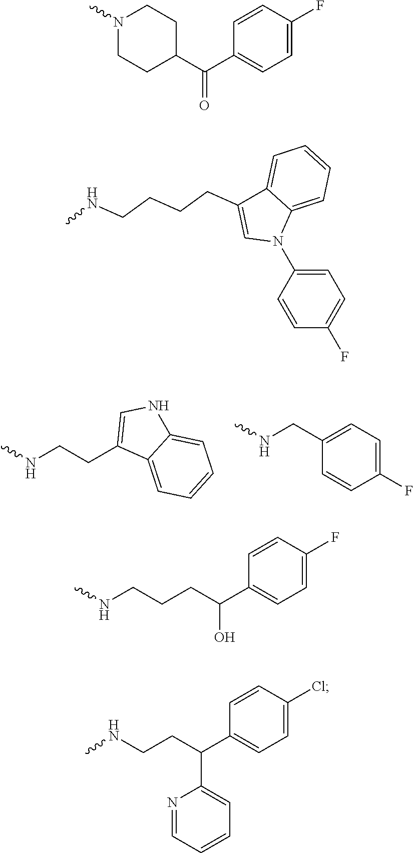

a compound of formula (III):

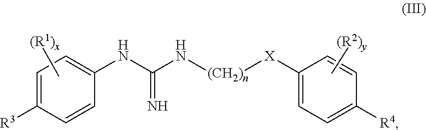

wherein:
each occurrence of R¹ and R² is independently selected from the group consisting of —C₁-C₆ alkyl, —C₁-C₆ fluoroalkyl, —C₁-C₆ heteroalkyl, F, Cl, Br, I, —CN, —NO₂, —OR⁵, —SR⁵, —S(═O)R⁵, —S(═O)₂R⁵, —NHS(═O)₂R⁵, —C(═O)R⁵, —OC(═O)R⁵, —CO₂R⁵, —OCO₂R⁵, —CH(R⁵)₂, —N(R⁵)₂, —C(═O)N(R⁵)₂, —OC(═O)N(R⁵)₂, —NHC(═O)NH(R⁵), —NHC(═O)R⁵, —NHC(═O)OR⁵, —C(OH)(R⁵)₂, and —C(NH₂)(R⁵)₂;
R³ is selected from the group consisting of —C₁-C₆ alkyl, —C₁-C₆ fluoroalkyl, —C₁-C₆ alkoxy, F, Cl, Br, and I;
R⁴ is selected from the group consisting of —C₁-C₆ alkyl, —C₁-C₆ alkoxy, F, Cl, Br, and I;
each occurrence of R⁵ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted;
X is selected from the group consisting of CH₂, C═O, or O;
n is an integer from 1-3;
x is an integer from 0-4; and
y is an integer from 0-4;
a salt, solvate, or N-oxide thereof; and any combinations thereof.

Embodiment 19: The method of embodiment 18, wherein the compound is selected from the group consisting of:
1-(3-(4-fluorophenoxy)propyl)-3-(4-iodophenyl)guanidine (Compound A);
1-(3-(4-fluorophenoxy)propyl)-3-(4-methoxyphenyl)guanidine (Compound B);
1-(n-propyl)-3-(4-iodophenyl)guanidine (Compound C);
1-(n-propyl)-3-(4-methoxyphenyl)guanidine (Compound D);
1-(3-(4-fluorophenoxy)propyl)-3-(4-trifluoromethylphenyl)guanidine (Compound F);
1-(3-(4-fluorophenoxy)propyl)-3-(4-chlorophenyl)guanidine (Compound G, also referred to as “CT-110” or “CT-189” herein);
   a salt, solvate or N-oxide thereof, and any combinations thereof.

Embodiment 20: The method of embodiment 18, wherein the compound is selected from the group consisting of:
1,3-bis(3-(4-fluorophenoxy)propyl)guanidine (Compound E);
1-(3-(4-fluorophenoxy)propyl)-3-(4-methyl-2-oxo-2H-chromen-7-yl)guanidine) (Compound H);
   a salt, solvate or N-oxide thereof, and any combinations thereof.

Embodiment 21: The method of any of embodiments 18-20, wherein the compound is administered as a pharmaceutical composition further comprising a pharmaceutically acceptable carrier.

Embodiment 22: The method of any of embodiments 18-21, wherein the subject is further administered at least one additional agent that treats, prevents, and/or ameliorates one of NAFDL, NASH, and/or HCC.

Embodiment 23: The method of any of embodiments 18-22, wherein the compound is administered by a route comprising oral, nasal, rectal, intravaginal, parenteral, buccal, sublingual or topical.

Embodiment 24: The method of any of embodiments 18-23, wherein the subject is a mammal.

Embodiment 26: The method of embodiment 24, wherein the mammal is a human.

### BRIEF DESCRIPTION OF THE DRAWINGS

For the purpose of illustrating the disclosure, certain embodiments of the disclosure are depicted in the drawings. However, the disclosure is not limited to the precise arrangements and instrumentalities of the embodiments depicted in the drawings.

FIGS. 1A-1B illustrate SIGMAR1 transcript expression patterns in healthy human liver and non-alcoholic fatty (NAFL) liver, in accordance with some embodiments. FIG. 1A: SIGMAR1 mRNA transcript expression levels in 27 organs (data from Fagerberg, Mol. Cell. Proteomics 2014; PMID: 24309898). FIG. 1B: SIGMAR1 mRNA transcription expression levels in healthy human livers and non-alcoholic fatty liver (NAFL) livers. Horizontal lines on scatter plot represent median Z-score.

FIGS. 2A-2E demonstrate that SIGMAR1 knockout (KO) mice are protected against diet-induced hepatic steatosis, in accordance with some embodiments. FIG. 2A depicts the steatosis grades of wild-type (WT) and SIGMAR1 KO mice fed with normal diet (ND) or high fat diet (HFD). Area of liver sections composed of lipid (% area) in normal diet and high fat diet wild-type and SIGMAR1 KO mice. FIG. 2B shows Hematoxylin & Eosin (H&E) staining images of livers from ND v HFD fed wild-type (WT), SIGMAR1 KO (σ1(−/−) mice. Images taken at 14×, scalebar=200 um. FIG. 2C depicts the liver steatosis grade calculated from the mice of FIG. 2A. FIG. 2D shows representative photos of livers from WT and SIGMAR1 KO (σ1(−/−)) mice fed with high fat diet. FIG. 2E shows Oil-red-O staining images of livers from WT and SIGMAR1 KO mice fed with high fat diet.

FIGS. 3A-3C demonstrate that high fat diet induced whitening of brown adipose tissue (BAT), in accordance with some embodiments. Lipid content in BAT of wild-type (WT) and SIGMAR1 KO mice fed high fat diet (HFD) for 8-weeks. FIG. 3A show images of representative fields from H&E-stained BAT of WT and SIGMAR1 KO male mice after 8-weeks of HFD. Images taken at 14×, scalebar=200 um. FIG. 3B depicts the lipid area on H&E-stained sections, 3 fields for each animal, n=4 animals for each group. Quantified using ImageJ. Statistical analysis used, one-way ANOVA Tukey's post-test, statistical significance of pair-wise comparisons are shown. FIG. 3C depicts average lipid droplet (LD) size in square microns (μM²). Quantified using Aperio software. Statistical analysis used, one-way ANOVA Tukey's post-test, statistical significance of pair-wise comparisons are shown.

FIGS. 4A-4B illustrate the finding that Sigma1 (σ1) knockdown by transduction of SIGMAR1 targeted shRNA using a lentiviral vector abrogates lipid droplet (LD) accumulation, in accordance with some embodiments. FIG. 4A: Confocal micrographs of LNCaP cells treated with 72 hours (72 h) with dihydrotestosterone (DHT) to induce the formation and accumulation of LDs. Non-specific Control shRNA was compared to SIGMAR1 transcript selective shRNAs in lentiviral vectors. σ1 shR #4=MISSION/SigmaAldrich clone TRCN0000061010: σ1 shR #5=MISSION/SigmaAldrich clone TRCN0000061008. White boxes indicate areas magnified and shown immediately below. Note nearly complete elimination of LDs. FIG. 4B: Immunoblot confirming knockdown of Sigma1 by lentiviral Sigma1 (σ1) shRNAs. LDs are stained with HCS-LipidTox fluorescent stain in red. Blue DAPI stained nuclei.

FIG. 5 illustrates the finding that pharmacological Sigma1 modulator/inhibitor 1-(4-Iodophenyl)-3-(2-adamantyl)guanidine (IPAG) eliminates lipid droplets (LDs), in accordance with some embodiments. Confocal micrographs of LNCaP cells treated with 72 hours (72 h) with 1 nM dihydrotestosterone (DHT) to induce the formation and accumulation of LDs. In the far right panel, the LNCaP cells were treated with a prototypic Sigma1 selective small molecule compound, IPAG (10 uM), for the final 16 hours of the 72 h DHT treatment period. LDs are stained with HCS-LipidTox fluorescent stain in red. Blue DAPI stained nuclei.

FIGS. 6A-6C demonstrate that the drug-like Sigma1 inhibitor (CT-110, also referred to as “CT-189” and “Compound G”) prevents diet-induced hepatic steatosis, in accordance with some embodiments. FIG. 6A illustrate certain aspects of the study design. As shown in FIG. 6A, high fat diet (HFD) fed mice were co-treated with either Vehicle or the novel drug-like small molecule Sigma1 inhibitor, CT-110, per os (PO) 3 days per week. FIG. 6B show H&E staining images of liver from mice in FIG. 6A. FIG. 6C shows images of brown adipose tissue (BAT) from mice on high fat diet (HFD) treated with either vehicle or CT-110 (also referred to as “CT-189” and “Compound G”). Representative images from H&E-stained BAT of mice after an 8-week HFD. Images taken at 14×, scalebar=200 um.

FIG. 7 illustrates the finding that Sigma1 inhibition with CT-189 suppresses levels of key fibrosis associated pro-inflammatory cytokines in HT-29 (a human colorectal adenocarcinoma cell line), in accordance with some embodiments. The fibrosis associated pro-inflammatory cytokines include soluble IL-6, TNFα, and IL-17, which are contributors to the progression of NAFLD to NASH with fibrosis.

### DETAILED DESCRIPTION

The studies described herein (“the present studies”) disclose, in one aspect, that the expression of Sigma 1 receptor is significantly increased in the livers of people with non-alcoholic fatty (NAFL) liver. Using SIGMAR1 knockout mice as a non-limiting example, the present studies discover that the elimination of Sigma I expression are able to substantially reduce the level of steatosis and whitening of brown adipose tissue in liver induced by a high fat diet (HFD). Using shRNA as a non-limiting example, the present studies discovered that reducing the expression level of Sigma1 is able to reduce DHT-induced formation and accumulation of lipid droplet in cells. Using the small molecule Sigma 1 inhibitor IPAG (1-(4-Iodophenyl)-3-(2-adamantyl)guanidine) as a non-limiting example, the present studies discovered that pharmaceutical inhibition of Sigma I receptor is able to reduce DHT-induced formation and accumulation of lipid droplet in cells. Using the small molecule Sigma 1 inhibitor CT-110 (also referred to as “CT-189” or “Compound G” herein) as a non-limiting example, the present studies discovered that pharmaceutical inhibition of Sigma I receptor in mice is able to reduce steatosis and whitening of brown adipose tissue in liver induced by a high fat diet. Furthermore, the present studies discovered that pharmaceutical inhibition of Sigma I receptor is able to reduce the expression level of pro-inflammatory cytokines associated with fibrosis, such as the fibrosis of liver which often happen following the nonalcoholic fatty liver disease.

The present disclosure relates, in one aspect, to the finding that pharmacological and/or genetic (i.e., gene therapy) modulation of Sigma I receptor (also known as Sigma1) in the liver is useful for treating and/or preventing nonalcoholic fatty liver disease (NAFLD), and/or preventing and/or ameliorating progression from fatty liver to nonalcoholic steatohepatitis with fibrosis to hepatocellular carcinoma (HCC). Further, according to the present disclosure, Sigma1 can act as an useful diagnostic, prognostic, and/or pharmacodynamic biomarker in any such condition and/or disease evolution.

The present disclosure establishes a physiological role for Sigma1 as a necessary factor in the progression of non-alcoholic fatty liver disease (NAFLD) to non-alcoholic steatohepatitis (NASH) and establishes Sigma1 as a novel target and biomarker in the progression of NAFL-to-NASH.

Sigma1 (gene name SIGMAR1, and also known as sigma-1 receptor, Sigma1R, and several other names; see PMID: 28744586, 28871306) is a novel ligand-operated intracellular, integral membrane protein enriched in the secretory pathway of most cells. Sigma receptors are distinct from classical opioid receptors. Sigma1 is highly conserved among mammals (greater than 80% amino acid identity), but shares no significant homology with any traditional receptor family or other mammalian protein. Cloned Sigma1 is a 26 kilo Dalton integral membrane protein found primarily in the ER, and can translocate to the plasma membrane, other organelles, and endoplasmic membrane microdomains. Sigma1 itself has no known intrinsic signaling or enzymatic activity, rather it allosterically modulates the intracellular signaling and activities of its associated proteins. Thus, Sigma1 appears to be a component of the cellular support machinery. Sigma1 can participate in ligand-induced cell death by lysosomal destabilization and oxidative stress.

Sigma1 physically associates with several oncogenic driver proteins and Sigma1 functions via protein-protein interactions (PPI) to regulate their stability, transport, and degradation in a pharmacologically controllable manner. Sigma1 also regulates lipid and protein homeostasis at multiple levels and plays a critical role in increased lipid and protein synthesis, processing, and quality control associated with tumor growth. Thus, targeting Sigma1 enables selective modulation of multiple nodes via one drug target. A consequence of the secretory pathway regulating properties of Sigma1 modulators is that they can be used to elicit immune modulatory and anti-inflammatory effects. Sigma1 regulates lipid metabolism, and intact Sigma1 is required by prostate cancer cells to utilize lipid droplet accumulation and metabolism for disease progression and malignancy.

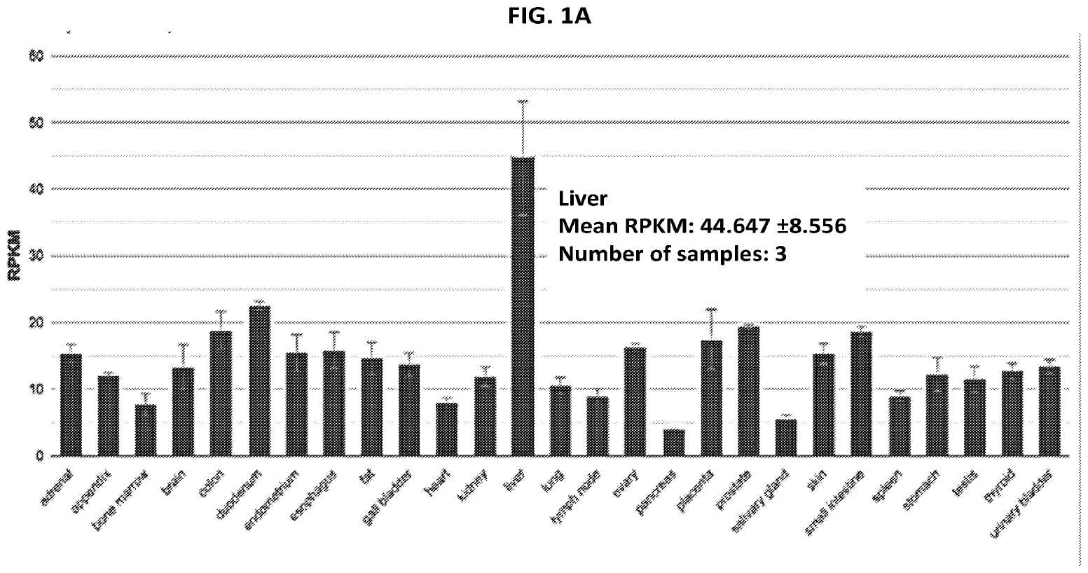

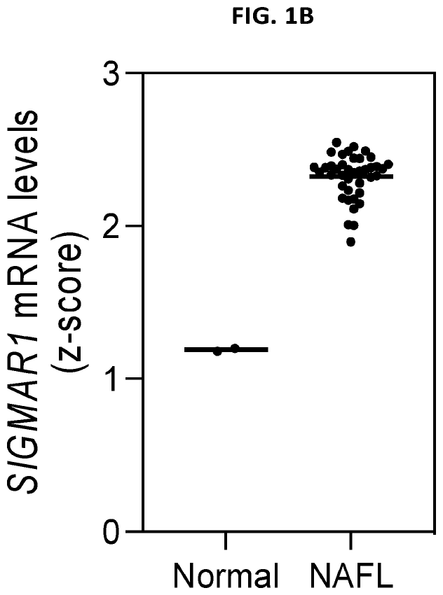

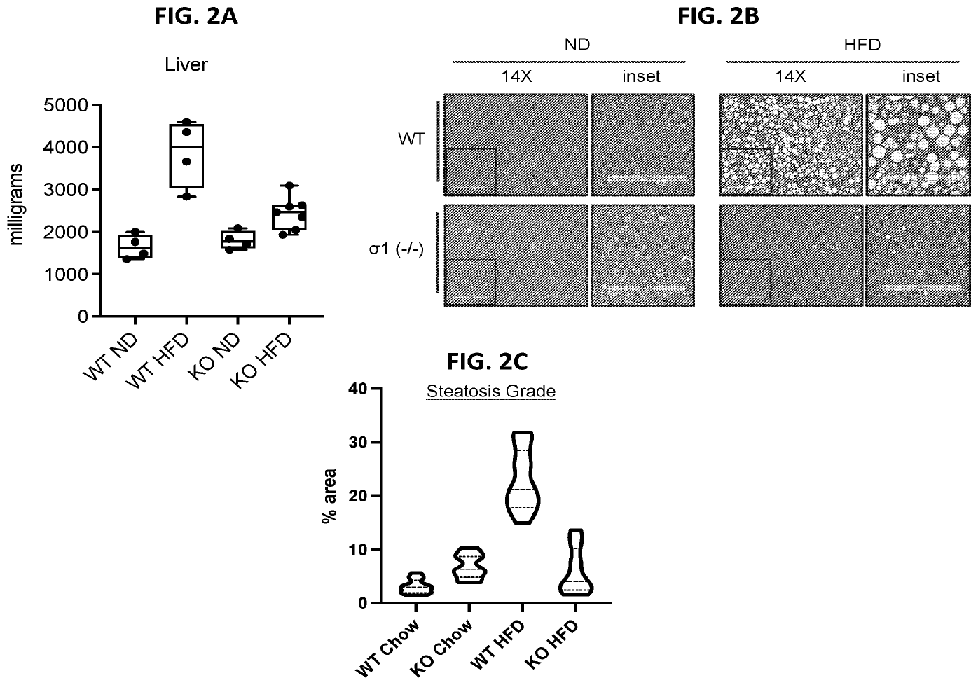

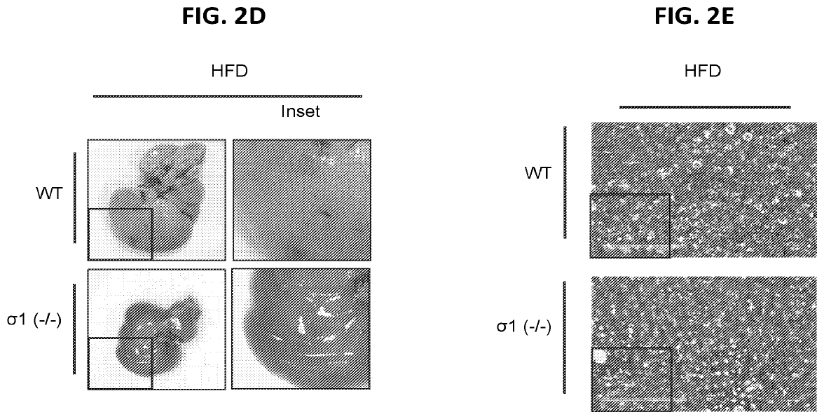

Importantly, Sigma1 is a multifunctional drug target that is engaged differently in pathophysiological and physiological conditions. The data provided herein show that SIGMAR1/Sigma1 plays a key role in NAFLD. Clearly higher levels of SIGMAR1 transcripts are observed in liver, but a role for SIGMAR1/Sigma1 in liver physiology and pathophysiology remains undefined. SIGMAR1 transcript levels are elevated in NAFL and fibrotic NASH livers compared to healthy liver and interestingly decrease sharply in cirrhotic liver (FIGS. 1A-1B). This indicates a changing role for Sigma1 as NAFLD progresses from NASH to HCC. High fat diet (HFD) does not induce liver steatosis in SIGMAR1 KO mice (FIGS. 2A-2C), indicating that Sigma1 is required for the development of NAFL. Further, Sigma1 inhibition suppresses levels of key fibrosis associated pro-inflammatory cytokines including soluble IL-6, TNFα, and IL-17, which are clear contributors to the progression of NAFLD to NASH.

In certain embodiments, Sigma1 is required for high fat western diet (HFWD) induced NAFL and progression to NASH and HCC. Thus, Sigma1 inhibition can be hepatoprotective and can prevent or delay progression of NAFLD to NASH by preventing hepatic steatosis and inflammation associated with steatohepatitis and progression to cirrhosis and HCC.

In certain embodiments, the progression from NAFL-to-NASH-to-HCC in high fat western diet (HFWD) fed SIGMAR1 knockout (KO) v. wild-type (WT) mice is characterized. In this study, the complete SIGMAR1 knockout (KO) C57BL/6 mouse model that is fed a HFWD+CCl₄ (Tsuchida, et al., 2018, J. Hepatol. 69:385-395) is used to evaluate key biomarkers of disease progression: steatosis, inflammation, hepatocyte ballooning, fibrosis, hepatic stellate cell (HSC) activation, serum liver enzymes, insulin resistance/glucose tolerance, and/or formation of HCC lesions. The status of SIGMAR1 transcripts and Sigma1 protein levels, as well as immunostaining patterns in liver at all stages of disease progression, are monitored on the WT C57BL/6 mice.

In certain embodiments, the impact of Sigma1 targeted pharmacotherapy in prevention and regression of steatosis and fibrosis is evaluated. In other embodiments, small molecule Sigma1 modulators phenocopy key aspects of the hepatoprotective effect of the SIGMAR1 KO mouse. One can then determine at which stage of disease progression Sigma1 pharmacotherapy can be effective. It can be determined whether Sigma1 pharmacotherapy can be used to prevent steatosis, inflammation, and/or fibrosis. This helps prove that Sigma1 pharmacotherapy is protective and prevent hepatic steatosis and fibrosis and/or can promote and accelerate recovery after onset of steatosis and after onset of fibrosis.

The present disclosure includes any compound contemplated within the disclosure, as well as compositions comprising the same, wherein the compositions optionally further comprise at least one additional therapeutic agent and/or at least one pharmaceutically acceptable carrier.

In certain embodiments, the compounds contemplated within the disclosure have improved drug-like properties over compounds known in the art to bind to and modulate the Sigma receptor. In another embodiment, the compounds contemplated within the disclosure do not cross the blood-brain barrier. In yet another embodiment, the compounds contemplated within the disclosure cross the blood-brain barrier.

The compounds contemplated within the disclosure can be characterized by pharmacological, cellular, biochemical, in vivo, pharmacokinetics, or pharmacodynamics properties. Selected examples of characterization studies include, but are not limited to, Sigma1-ligand binding properties, signaling pathway analysis and/or characterization, proteomic analysis of Sigma1 protein associations in response to Sigma ligand treatment, tumor, brain response, and toxicity.

The entire disclosures of WO 2014/015157 and US 2015/0166472 are incorporated herein in their entireties by reference.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” or “ameliorated” if the severity of a symptom of the disease or disorder, the frequency with which such a symptom is experienced by a patient, or both, is reduced.

As used herein, the term “Sigma” refers to the Sigma1 receptor (Sigma1), Sigma2 receptor (Sigma2), any splice variant thereof or any isoform thereof. The canonical form of Sigma1 has the amino acid sequence of SEQ ID NO:1. It should be noted that amino acids 2-8 target the protein to endoplasmic reticulum-associated lipid droplets; amino acids 99-106 are important for ligand-binding; amino acids 177-223 correspond to the C-terminal hydrophobic region.

```
                10 20 30 40
MQWAVGRRWA WAALLLAVAA VLTQVVWLWL GTQSFVFQRE
                50 60 70 80
EIAQLARQYA GLDHELAFSR LIVELRRLHP GHVLPDEELQ
                90 100 110 120
WVFVNAGGWM GAMCLLHASL SEYVLLFGTA LGSRGHSGRY
              130 140 150 160
WAEISDTIIS GTFHQWREGT TKSEVFYPGE TVVHGPGEAT
              170 180 190 200
AVEWGPNTWM VEYGRGVIPS TLAFALADTV FSTQDELTLF
              210 220
YTLRSYARGL RLELTTYLFG QDP
```

As used herein, a “Sigma receptor modulator” is a compound that binds to the Sigma receptor and modifies the activity or biological function of the receptor as compared to the activity or biological function of the receptor in the absence of the modulator. In certain embodiments, the modulator may be able to activate the receptor and thus cause a biological response that is enhanced over the baseline activity of the unbound receptor. In certain embodiments, the modulator may not activate the receptor thoroughly and thus cause a biological response that is smaller in magnitude compared to those of full modulators. In certain embodiments, the modulator may bind to the receptor but not activate it, resulting in receptor blockage and inhibiting the binding of other modulators. Such an modulator does not diminish the baseline intracellular response in the absence of an modulator. In certain embodiments, the modulator may function as a putative antagonist, agonist, or as an inverse agonist, which reduces the activity of the receptor by inhibiting its constitutive activity.

As used herein, the term “Sigma1i” refers to a Sigma receptor modulator. “Sigma1i”=Sigma1 inhibitor. “Sigma1a”=Sigma1 activator. Sigma1 modulator=any compound that binds/has affinity for Sigma1 and triggers activity.

The terms “patient,” “subject,” “individual,” and the like are used interchangeably herein, and refer to any animal, or cells thereof whether in vitro or in situ, amenable to the methods described herein. In a non-limiting embodiment, the patient, subject or individual is a human.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the disclosure (alone or in combination with another pharmaceutical agent), to a patient, or application or administration of a therapeutic agent to an isolated tissue or cell line from a patient (e.g., for diagnosis or ex vivo applications), who has a condition contemplated herein and/or a symptom of a condition contemplated herein, with the purpose to cure, heal, alleviate, relieve, alter, remedy, ameliorate, improve, or affect a condition contemplated herein and/or the symptoms of a condition contemplated herein. Such treatments may be specifically tailored or modified, based on knowledge obtained from the field of pharmacogenomics.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

An “effective amount” of a delivery vehicle is that amount sufficient to effectively bind or deliver a compound.

As used herein, the term “efficacy” refers to the maximal effect (Eₘₐₓ) achieved within an assay.

As used herein, the term “haloperidol” refers to 4-[4-(4-chlorophenyl)-4-hydroxy-1-piperidyl]-1-(4-fluorophenyl)-butan-1-one, or a salt or solvate thereof.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “potency” refers to the dose needed to produce half the maximal response (ED₅₀).

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NHOCH₃, or —CH₂CH₂SSCH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In certain embodiments, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

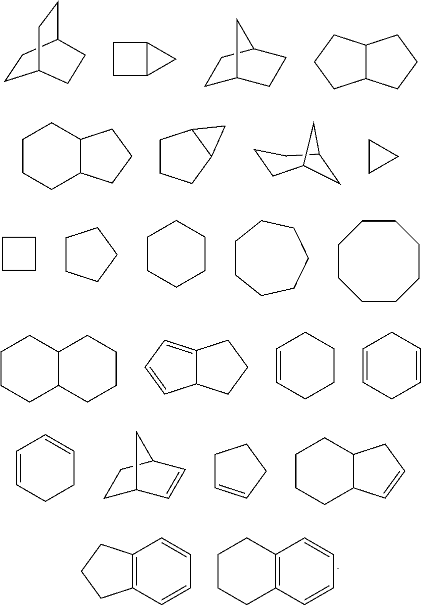

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, Sand N. In certain embodiments, each heterocycloalkyl group has from 4 to 10 atoms in its ring system, with the proviso that the ring of said group does not contain two adjacent O or S atoms. In another embodiment, the heterocycloalkyl group is fused with an aromatic ring. In certain embodiments, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

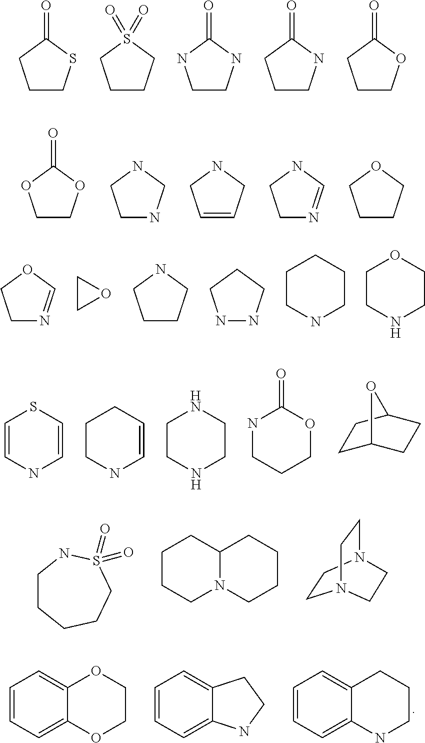

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

As used herein, the term “aryl-(C₁-C₃)alkyl” means a functional group wherein a one to three-carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heteroaryl” or “heteroaromatic” refers to a heterocycle having aromatic character. A polycyclic heteroaryl may include one or more rings that are partially saturated. Examples include the following moieties:

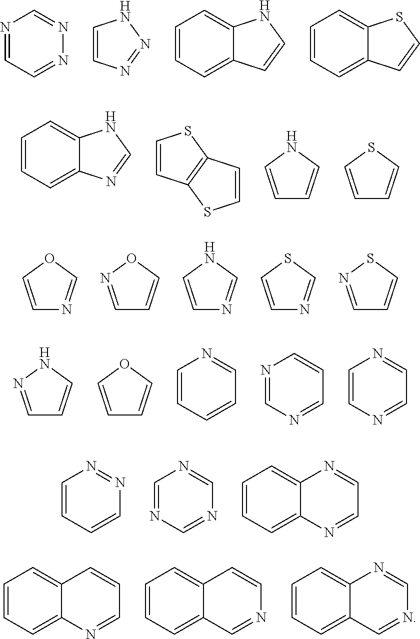

Examples of heteroaryl groups also include pyridyl, pyrazinyl, pyrimidinyl (particularly 2- and 4-pyrimidinyl), pyridavinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In certain embodiments, the referenced group is optionally substituted with zero substituents, i.e., the referenced group is unsubstituted. In another embodiment, the referenced group is optionally substituted with one or more additional group(s) individually and independently selected from groups described herein.

In certain embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

The compounds contemplated within the disclosure may be synthesized using techniques well-known in the art of organic synthesis. The starting materials and intermediates required for the synthesis may be obtained from commercial sources or synthesized according to methods known to those skilled in the art.

In one aspect, the compound contemplated within the disclosure is a compound of formula (I), or a salt, solvate, or N-oxide thereof:

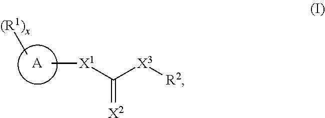

wherein:
ring A is a monocyclic or bicyclic aryl or a monocyclic or bicyclic heteroaryl ring, and wherein the aryl or heteroaryl ring is optionally substituted with 0-4 R¹ groups;
each occurrence of R¹ is independently selected from the group consisting of —C₁-C₆ alkyl, —C₁-C₆ fluoroalkyl, —C₁-C₆ heteroalkyl, F, Cl, Br, I, —CN, —NO₂, —OR³, —SR³, —S(═O)R³, —S(═O)₂R³, —NHS(═O)₂R³, —C(═O)R³, —OC(═O)R³, —CO₂R³, —OCO₂R³, —CH(R³)₂, —N(R³)₂, —C(═O)N(R³)₂, —OC(═O)N(R³)₂, —NHC(═O)NH(R³), —NHC(═O)R³, —NHC(═O)OR³, —C(OH)(R³)₂, and —C(NH₂)(R³)₂;
each occurrence of R² is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl or cycloalkyl group is optionally substituted with 0-5 R¹ groups, or X³ and R² combine to form a (C₃-C₇)heterocycloalkyl group, optionally substituted with 0-2 R¹ groups;
each occurrence of R³ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted with 0-5 R¹ groups;
X¹ is —CH₂—, —S—, —O— or —(NR₂)—;
X² is ═CH₂, ═S, ═O or ═NR₂; and
X³ is —S—, —O—, or —NR₂—.

In certain embodiments, ring A is a monocyclic aryl or monocyclic heteroaryl ring optionally substituted with 0-4 R¹ groups. In another embodiment, ring A is unsubstituted. In yet another embodiment, ring A is phenyl or substituted phenyl.

In a preferred embodiment, X¹ and X³ are both-NH—, and X² is ═NH.

In another aspect, the compound contemplated within the disclosure is a compound of formula (II), or a salt, solvate, or N-oxide thereof:
R^{A}—R^{B} (II), wherein;
R^{A} is selected from the group consisting of

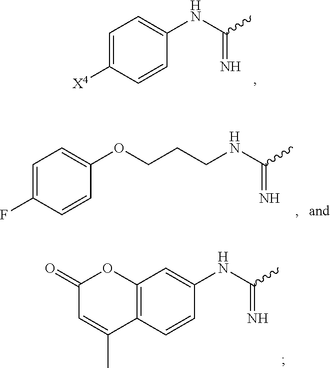

wherein
X⁴ is selected from the group consisting of methoxy, F, Cl, Br, and I; and
R^{B} is selected from the group consisting of:

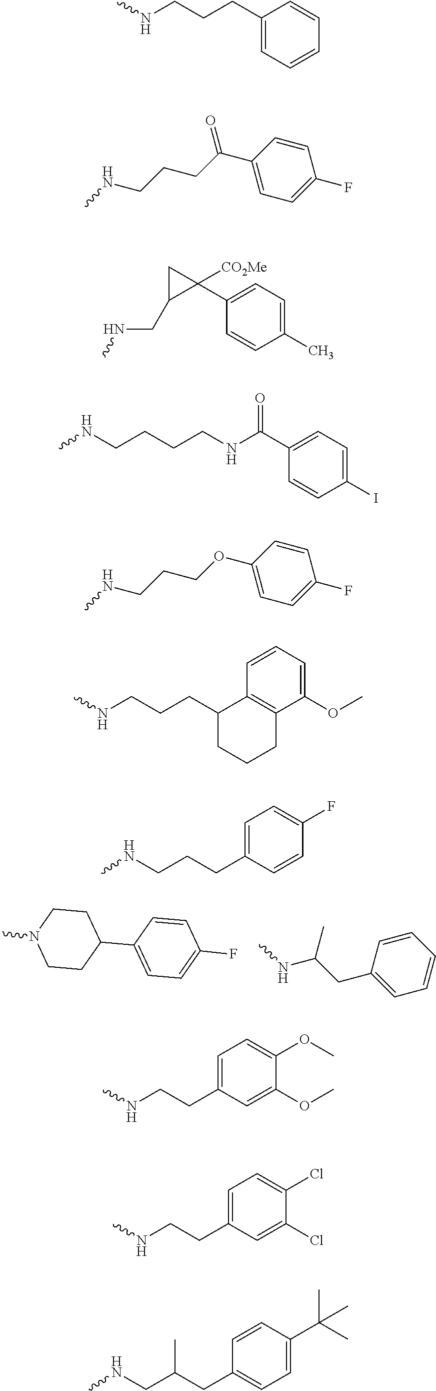

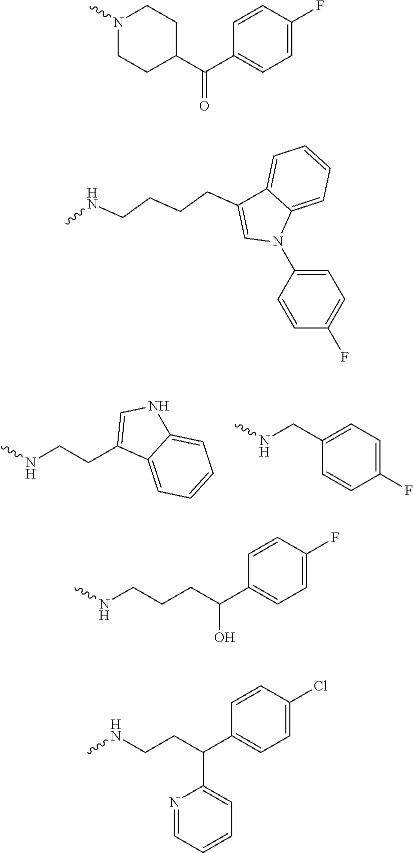

In another aspect, the compound contemplated within the disclosure is a compound of formula (III), or a salt, solvate, or N-oxide thereof:

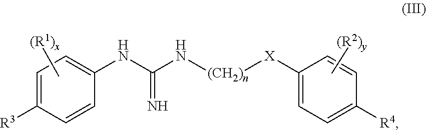

wherein:
each occurrence of R¹ and R² is independently selected from the group consisting of —C₁-C₆ alkyl, —C₁-C₆ fluoroalkyl, —C₁-C₆ heteroalkyl, F, Cl, Br, I, —CN, —NO₂, —OR⁵, —SR⁵, —S(═O)R⁵, —S(═O)₂R⁵, —NHS(═O)₂R⁵, —C(═O)R⁵, —OC(═O)R⁵, —CO₂R⁵, —OCO₂R⁵, —CH(R⁵)₂, —N(R⁵)₂, —C(═O)N(R⁵)₂, —OC(═O)N(R⁵)₂, —NHC(═O)NH(R⁵), —NHC(═O)R⁵, —NHC(═O)OR⁵, —C(OH)(R⁵)₂, and —C(NH₂)(R⁵)₂;
R³ is selected from the group consisting of —C₁-C₆ alkyl, —C₁-C₆ fluoroalkyl, —C₁-C₆ alkoxy, F, Cl, Br, and I;
R⁴ is selected from the group consisting of —C₁-C₆ alkyl, —C₁-C₆ alkoxy, F, Cl, Br, and I;
each occurrence of R⁵ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted.
X is selected from the group consisting of CH₂, C═O, or O;
n is an integer from 1-3;
x is an integer from 0-4; and
y is an integer from 0-4.

In certain embodiments, the compound contemplated within the disclosure is selected from the group consisting of:
1-(3-(4-fluorophenoxy)propyl)-3-(4-iodophenyl)guanidine (Compound A; also known as JMS-51-58 or 51-58);
1-(3-(4-fluorophenoxy)propyl)-3-(4-methoxyphenyl)guanidine (Compound B);
1-(n-propyl)-3-(4-iodophenyl)guanidine (Compound C);
1-(n-propyl)-3-(4-methoxyphenyl)guanidine (Compound D);
1,3-bis(3-(4-fluorophenoxy)propyl)guanidine (Compound E);
1-(3-(4-fluorophenoxy)propyl)-3-(4-trifluoromethylphenyl)guanidine (Compound F);
1-(3-(4-fluorophenoxy)propyl)-3-(4-chlorophenyl)guanidine (Compound G, also referred to as CT-189 or CT-110 herein);
1-(3-(4-fluorophenoxy)propyl)-3-(4-methyl-2-oxo-2H-chromen-7-yl)guanidine (Compound H);
   a salt, solvate or N-oxide thereof; and any combinations thereof.

In some aspects, the compound contemplated within the disclosure includes 1-(4-Iodophenyl)-3-(2-adamantyl)guanidine (IPAG).

### Preparation of the Compounds

Compounds contemplated within the disclosure may be prepared by the general schemes described herein, using the synthetic method known by those skilled in the art. The following examples illustrate non-limiting embodiments of the disclosure.

In a non-limiting embodiment, the synthesis of unsymmetrical N,N′-disubstituted guanidines is accomplished by coupling an aryl cyanamide and an amine. In certain embodiments, the coupling reaction takes place at an elevated temperature ranging from 80° C. to 250° C. An aniline may be converted to an aryl cyanamide with cyanogen bromide in ether. The unsymmetrical N,N′-disubstituted guanidine is then formed by coupling the aryl cyanamide with an amine. Non-limiting examples of coupling methods include heating in acetonitrile at reflux, and heating at 120° C. in a microwave.

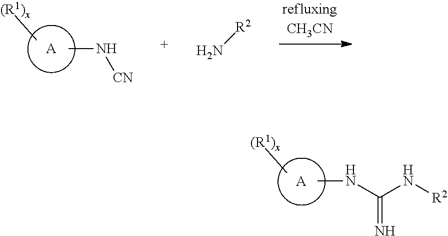

In another non-limiting embodiment, unsymmetrical N,N′-disubstituted guanidines may be synthesized by coupling a benzimidothioate and an amine. For example, an aniline may be reacted with potassium isothiocyanate to provide a thiourea. The thiourea may then be treated with methyl iodide in acetone heated to reflux, providing the desired benzimidothioate. The unsymmetrical N,N′-disubstituted guanidine may then be formed by coupling the benzimidothioate with an amine. A non-limiting example of a coupling method includes heating in ethanol at reflux.

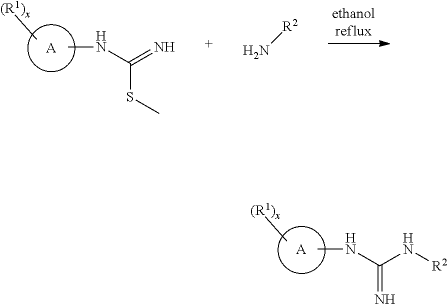

The compounds of the disclosure may possess one or more stereocenters, and each stereocenter may exist independently in either the R or S configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In another embodiment, the compounds described herein exist in unsolvated form.

In certain embodiments, the compounds of the disclosure may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In another embodiment, a prodrug is enzymatically metabolized by one or more steps or processes to the biologically, pharmaceutically or therapeutically active form of the compound.

In certain embodiments, sites on, for example, the aromatic ring portion of compounds of the disclosure are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

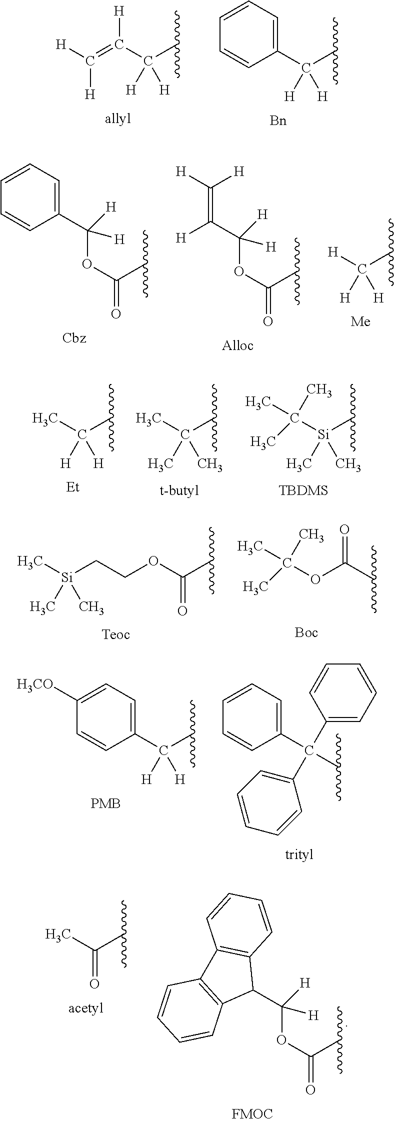

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Nucleic Acid Inhibitors/Modulator

In certain embodiments, the Sigma1 modulating agent comprises an isolated nucleic acid. In other embodiments, the modulator is an RNAi molecule (such as but not limited to siRNA and/or shRNA and/or miRNAs) or antisense molecule, which inhibits Sigma1 expression and/or activity. In yet other embodiments, the nucleic acid comprises a promoter/regulatory sequence such that the nucleic acid is preferably capable of directing expression of the nucleic acid. Thus, the disclosure provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, Sigma1 can be inhibited by way of inactivating and/or sequestering Sigma1. As such, inhibiting the activity of Sigma1 can be accomplished by using a transdominant negative mutant.

In certain embodiments, siRNA is used to decrease the level of Sigma1. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19): 306-311; Timmons et al., 1998, Nature 395:854; Montgomery et al., 1998, TIG 14 (7):255-258; Engelke, Ed., RNA Interference (RNAi) Nuts & Bolts of RNAi Technology, DNA Press, Eagleville, PA (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present disclosure also includes methods of decreasing levels of Sigma1 using RNAi technology.

In certain embodiments, the disclosure provides a vector comprising an siRNA or antisense polynucleotide. In other embodiments, the siRNA or antisense polynucleotide inhibits the expression of Sigma1. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the disclosure relates to a vector, comprising the nucleotide sequence of the disclosure or the construct of the disclosure. The choice of the vector will depend on the host cell in which it is to be subsequently introduced. In certain embodiments, the vector of the disclosure is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

Further, the expression vector may be provided to a cell in the form of a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

The viral vector can be any suitable adeno-associated virus (AAV), such as the AAV1-AAV8 family of adeno-associated viruses. In some embodiments, the viral vector is a viral vector that can infect a human. The desired nucleic acid sequence can be inserted between the inverted terminal repeats (ITRs) in the AAV. In various embodiments, the viral vector is an AAV8. The promoter can be a thyroxine binding globulin (TBG) promoter. In various embodiments, the promoter is a human promoter sequence that enables the desired nucleic acid expression in the liver. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the disclosure contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein.

By way of illustration, the vector in which the nucleic acid sequence is introduced can be a plasmid that is or is not integrated in the genome of a host cell when it is introduced in the cell. Illustrative, non-limiting examples of vectors in which the nucleotide sequence of the disclosure or the gene construct of the disclosure can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of disclosure, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring.” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide has certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

In certain embodiments, an antisense nucleic acid sequence expressed by a plasmid vector is used to inhibit Sigma1 protein expression. The antisense expressing vector is used to transfect a mammalian cell or the mammal itself, thereby causing reduced endogenous expression of Sigma1.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the disclosure may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the disclosure include oligonucleotide derivatives known in the art which have improved biological activity compared to unmodified oligonucleotides (see U.S. Pat. No. 5,023,243).

In certain embodiments, a ribozyme is used to inhibit Sigma1 protein expression. Ribozymes useful for inhibiting the expression of a target molecule may be designed by incorporating target sequences into the basic ribozyme structure which are complementary, for example, to the mRNA sequence encoding Sigma1. Ribozymes targeting Sigma1, may be synthesized using commercially available reagents (Applied Biosystems, Inc., Foster City, CA) or they may be genetically expressed from DNA encoding them.

### CRISPR/Cas9

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9) protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present disclosure. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present disclosure may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

### Methods

The disclosure includes a method of treating, preventing, and/or ameliorating accumulation of lipid droplets (LDs) in a cell. The disclosure further includes a method of treating, preventing, and/or ameliorating hepatic steatosis and/or inflammation associated with steatohepatitis in a subject. The disclosure includes a method of treating, preventing, and/or ameliorating nonalcoholic fatty liver disease (NAFLD) in a subject. The disclosure also includes a method of reversing, slowing, or preventing progression of NAFLD to nonalcoholic steatohepatitis (NASH). The disclosure also includes a method of reversing, slowing, or preventing progression of NASH to hepatocellular carcinoma (HCC). The disclosure also includes a method of determining and/or evaluating risk that a subject suffering from NAFLD will progress to NASH. The disclosure also includes a method of determining and/or evaluating risk that a subject suffering from NASH will progress to HCC.

In certain embodiments, the method comprises administering to the subject an effective amount of at least one compound and/or composition contemplated within the disclosure. In other embodiments, the subject is further administered at least one additional agent that treats or prevents a disease and/or disorder contemplated herein. In yet other embodiments, the compound and the at least one additional agent are co-administered to the subject. In yet other embodiments, the compound and the at least one additional agent are co-formulated.

In certain embodiments, the subject is a mammal. In other embodiments, the mammal is a human.

### Combination Therapies

The compounds contemplated within the disclosure are intended to be useful in combination with one or more additional compounds. These additional compounds may comprise compounds of the present disclosure and/or at least one additional antiviral agent and/or at least one additional agent that treats one or more diseases or disorders contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70) mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of Sigma-receptor related disorders or diseases in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. patent application Ser. Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of Sigma-receptor related disorders or diseases in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday.”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The examples described herein illustrate aspects of the present disclosure. However, they are in no way a limitation of the teachings or disclosure of the present disclosure as set forth herein.

The examples described herein are provided for purposes of illustration only, and are not intended to be limiting unless otherwise specified. Thus, the disclosure should in no way be construed as being limited to the examples described herein, but rather, should be construed to encompass any and all variations which become evident as a result of the teaching provided herein.

Without further description, it is believed that one of ordinary skill in the art can, using the description and illustrative examples, make and utilize the compounds of the present disclosure and practice the claimed methods. The working examples therefore, specifically point out selected embodiments of the present disclosure, and are not to be construed as limiting in any way the remainder of the disclosure.

### EXPERIMENTAL EXAMPLES

The disclosure is now described with reference to the following Examples. These Examples are provided for the purpose of illustration only, and the disclosure is not limited to these Examples, but rather encompasses all variations that are evident as a result of the teachings provided herein.

The materials and methods employed in these experiments are now described.

### Example 1: Increased SIGMAR1 mRNA Level is Associated with Non-Alcoholic Fatty Liver

A role for SIGMAR1/Sigma1 in liver physiology and pathophysiology has been unclear. However, data presented herein proves that SIGMAR1/Sigma1 plays a central role in liver metabolism and stress response. SIGMAR1 transcript levels are remarkably 2-to-9 fold higher in liver compared to other organs (FIG. 1A). SIGMAR1 transcript levels are elevated >2-fold in fibrotic NASH livers compared to normal, healthy liver (FIG. 1B). Interestingly, SIGMAR1 transcript levels decrease sharply as fibrotic NASH livers progress to cirrhosis and HCC (FIG. 1B). In certain embodiments, this indicates a changing role for Sigma1 as NAFLD progresses from NASH to HCC.

On-target actions of Sigma1 modulators do not induce adverse effects (including weight loss and behavioral abnormalities), and selective Sigma1 antagonists/inhibitors do not show cytoxicity in animal studies. In fact, SIGMAR1 knockout (KO) mice are viable, fertile, and do not display a phenotype overtly different from wild type mice (Langa, et al., 2003, Eur J Neurosci 18:2188-2196), indicating that inhibiting Sigma1 has minimal impact on normal tissues. Thus, Sigma1 targeting drugs can be safely and effectively used as single agents and also can be used to supplement or enhance therapeutic efficacy.

### Example 2: SIGMAR1 Knockout Protects Against High Fat Diet-Induced Fatty Liver

In certain embodiments, the progression from NAFL-to-NASH-to-HCC in high fat western diet (HFWD) fed SIGMAR1 knockout (KO) v. wild-type (WT) mice was characterized. In this study, the complete SIGMAR1 knockout (KO) C57BL/6 mouse model fed a HFWD+CCl₄ (Tsuchida, et al., 2018, J Hepatol 69:385-395) is used to evaluate key biomarkers of disease progression: steatosis, inflammation, hepatocyte ballooning, fibrosis, hepatic stellate cell (HSC) activation, serum liver enzymes, insulin resistance/glucose tolerance, and HCC lesion formation. Status of SIGMAR1 transcripts and Sigma1 protein levels, as well as immunostaining patterns in liver at all stages of disease progression, are monitored in the WT C57BL/6 mice.

A complete SIGMAR1 knockout (KO) C57BL/6 mouse has been generated. C57BL/6 mice are a mouse strain for diet induced obesity and NAFLD studies. Two-year-old male wild-type SIGMAR1 and SIGMAR1 KO C57BL/6 mice were fed either normal diet (ND) or high fat diet (HFD) (FIGS. 2A-2E) for 8 weeks, then livers were harvested and evaluated for weight, histology, and steatosis. By 8 weeks on HFD, the livers of WT mice had median weight of 3.9 grams compared to 2.2 grams for SIGMAR1 KO mouse livers (FIG. 2A). In contrast, the weight of the livers of WT mice and SIGMAR1 KO mice fed with normal diet (ND) were comparable (FIG. 2A). Body weights were similar at approximately 56 grams for both WT and SIGMAR1 KO mice. Livers from HFD fed WT and SIGMAR1 KO mice were visibly different in size, color, and texture (FIG. 2D). Hematoxylin and eosin (H&E) stain showed markedly distinct tissue architecture with large empty spaces, which is indicative of lipid droplets as well as evidence of increased immune cell infiltration in the WT mouse livers (FIGS. 2B-2C). Neutral lipid/lipid droplet accumulation was confirmed with oil-Red-O stain, which showed saliently elevated levels of neutral lipids in HFD fed WT mouse livers (FIG. 2E).

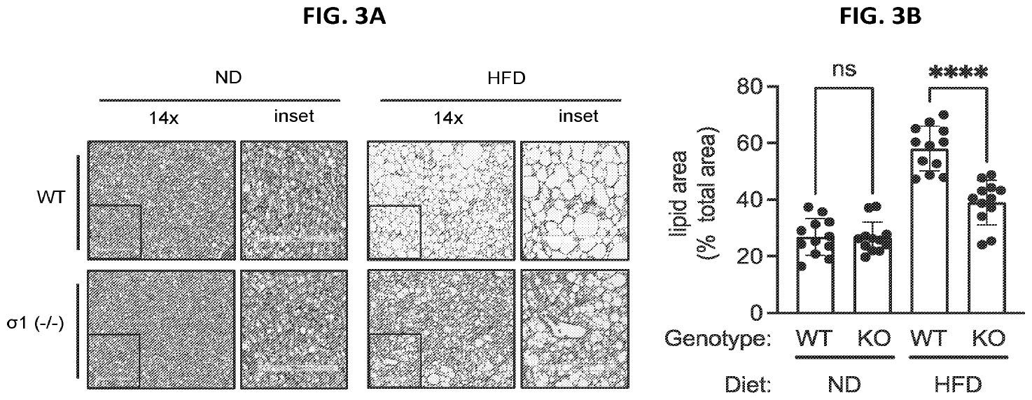

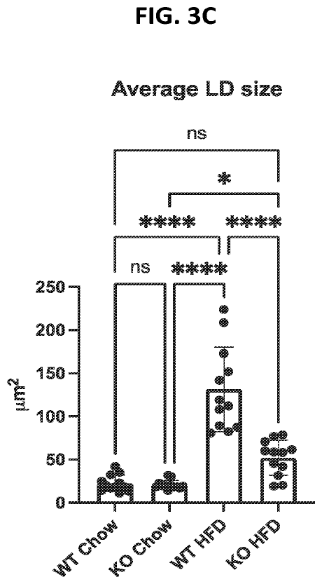

In mammals, white adipose tissue (WAT) stores and releases lipids, whereas brown adipose tissue (BAT) oxidizes lipids to fuel thermogenesis. In obese individuals, BAT content and activity decline as a result of the conversion of brown adipocytes to white-like unilocular cells. As shown in FIGS. 3A-3C, in wild-type mice, high fat diet (HFD) induced whitening of brown adipose tissue (BAT) (FIG. 3A), which involves both increased total lipid area (FIG. 3B) and increased average lipid droplet size (FIG. 3C). SIGMAR1 KO significantly rescued the whitening (FIG. 3A), lipid area increase (FIG. 3B) and lipid droplet size increase (FIG. 3C) caused by the HFD.

| | Normal Diet: | High Fat Diet: |
|---|---|---|
| | Lab Diet 5010 | TD.06414 |
| % kCal from Protein | 28.7% | 18.3% |
| % kCal from Fat | 13.1% | 60.3% |
| % kCal from Carbohydrates | 58.2% | 21.4% |

### Example 3: Down-Regulation of Sigma1 Expression Reduced Accumulation of Lipid Droplet in Cells

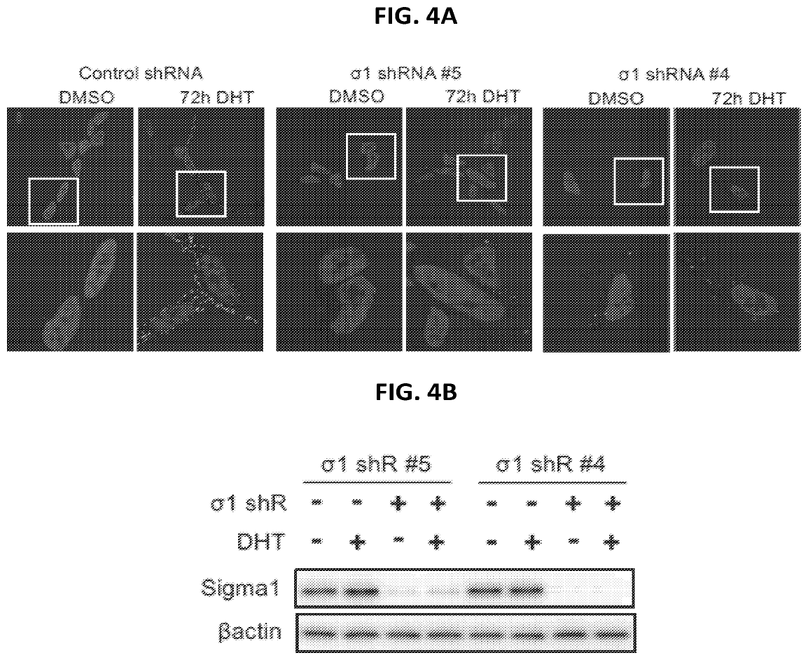

Referring to FIGS. 4A-4B, the present studies discover that down-regulation of Sigma1 (σ1) expression with SIGMAR1 targeted shRNA significantly reduced the accumulation of lipid droplets in cells. The shRNA was delivered using a lentiviral vector. As shown in FIG. 4B, two shRNAs, shRNA #4 (al shR #4=MISSION/SigmaAldrich clone TRCN0000061010) and shRNA #5 (σ1 shR #5=MISSION/SigmaAldrich clone TRCN0000061008), were confirmed to be able to down-regulate the expression of Sigma1. As shown in FIG. 4A, LNCaP cells treated with 72 hours (72 h) with dihydrotestosterone (DHT) (as well as a non-specific control shRNA) resulted in significant formation and accumulation of lipid droplets. Both shRNA #4 and shRNA #5 that specifically reduce Sigma1 expression, however; nearly complete eliminated the dihydrotestosterone induced LD formation and accumulation.

### Example 4: Pharmaceutical Inhibition of Sigma1 Reduced Level of High Fat Diet-Induced Liver Steatosis

The present studies evaluated the impact of Sigma1 targeted pharmacotherapy in prevention and regression of steatosis. In certain embodiments, small molecule Sigma1 modulators phenocopy key aspects of the hepatoprotective effect of the SIGMAR1 KO mouse.

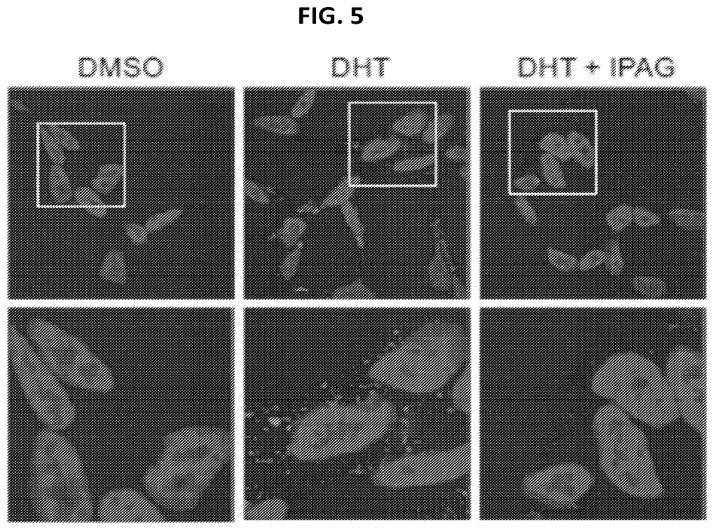

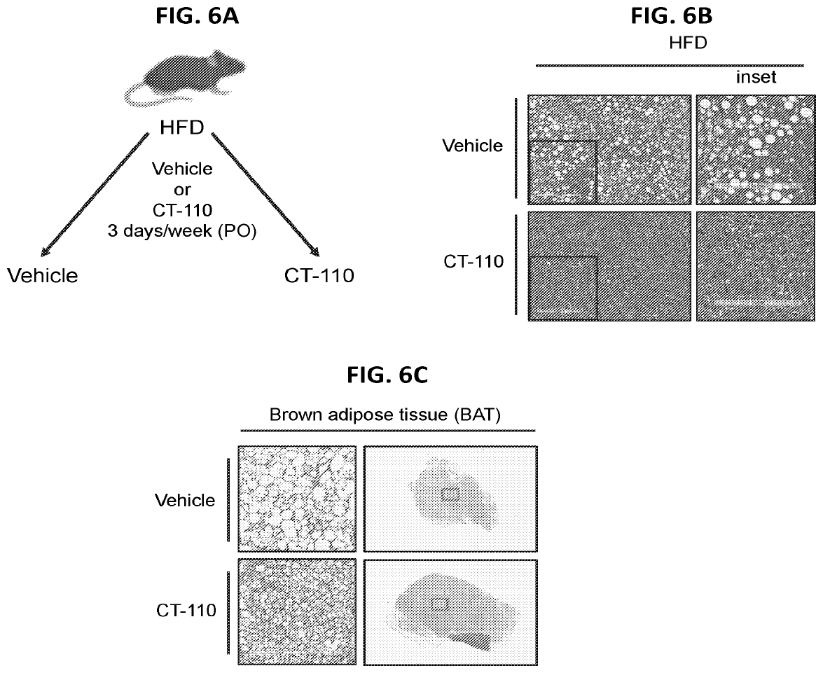

Refer to FIGS. 5 and 6A-6C, pharmacological inhibition of Sigma1 phenocopy certain aspects of Sigma1 knockdown. Oleic acid and glucose induced lipid droplets do not form in SIGMAR1 KO MEFs. The small molecule inhibitor IPAG also eliminate oleic acid, cholesterol, and glucose induced lipid droplets by lipophagy (FIG. 5). Referring to FIGS. 6A-6C, HFD fed mice orally administrated with the small molecule inhibitor CT-189 (also referred to as “Compound G” or “CT-110” herein), in comparison to mice administered with vehicle, showed significantly reduced level of liver fat (FIG. 6B), as well as significantly reduced level of whitening of brown adipose tissue (FIG. 6C).

The Sigma1i lead compound, CT189 [1-(4-chlorophenyl)-3-(3(4-fluorophenoxy)propyl)guanidine, also referred to herein as Compound G or CT-110] is orally bioavailable, binds Sigma1 with high affinity (Kᵢ=38 nM), and has limited off-target liabilities based on a CEREP Safety 44 panel screen. CT189 is safe and does not induce liver toxicity, even at doses that exceed the concentrations proposed herein (Salvino et al 2017 (PMID: 28385503); Thomas et al 2017 (PMID: 28235766); Kim and Maher 2017 (PMID: 28744586)). CT189 has been used in tumor xenograft experiments wherein daily oral dosing extended beyond 4 weeks and produced no detectable changes in mouse body weight and behavior. Therefore, this compound, through its inhibition of Sigma1 can elicit dual anti-neoplastic and steatosis reducing properties.

### Example 5: Pharmaceutical Inhibition of Sigma1 Reduces Expressions of Fibrosis Associated Pro-Inflammatory Cytokines

Often NASH patients are identified/diagnosed after they already have fibrotic disease. The present studies help determine whether Sigma1 pharmacotherapy can block the progression of hepatic fibrosis.

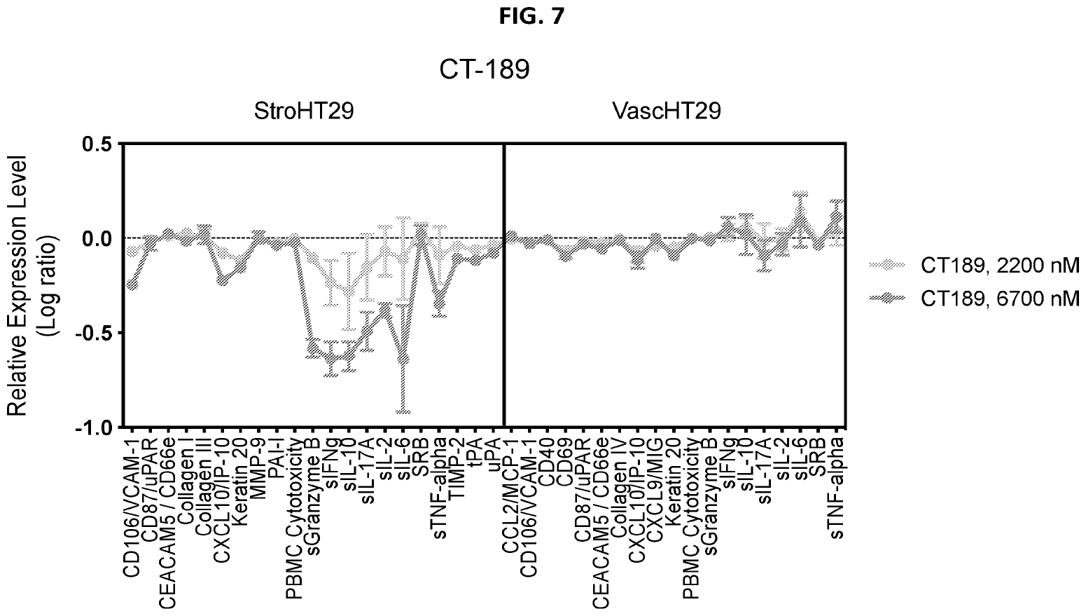

Referring to FIG. 7, Sigma1 inhibition suppresses levels of key fibrosis associated pro-inflammatory cytokines including soluble IL-6, TNFα, and IL-17 (FIG. 7). Sigma1 modulators have immunomodulatory and anti-inflammatory as well as anti-fibrotic properties. Further, some Sigma1 inhibitors suppress HCC cell proliferation and trigger apoptosis in vitro.

The present studies indicate whether Sigma1 targeted pharmacotherapy can stop NAFLD progression and whether it can accelerate regression and recovery after stopping HFWD after the onset of steatosis and/or NASH with fibrosis. One can determine whether Sigma1i drug can be used to enhance/promote recovery from steatosis and/fibrosis after diet change.

### Example 6

Steatosis. The present studies indicate whether Sigma1 targeted pharmacotherapy attenuates/mitigates steatosis while mice are still on HFWD. One can determine whether Sigma1 targeted pharmacotherapy accelerates and enhances the extent of regression of steatosis after mice have been placed back on normal diet (ND). Salient steatosis develops between 4-8 weeks on HFD. The data obtained herein shows elimination of high fat diet induced lipid droplets (FIGS. 2A-2E). Changes in steatosis are measured using oil-red-O and H&E stain as described elsewhere herein (FIGS. 2A-2E).

Inflammation. Several Sigma1 ligands have anti-inflammatory properties, and the in vitro data with CT189 demonstrate that this compound can suppress IL-17, IL-6, and TNFα, thus indicating that this compound series can effectively suppress inflammation in the liver (FIG. 7). To test whether Sigma1i can prevent inflammation in fatty liver, treatment starts at week 4-8 and continues to week 8-12. To test whether Sigma1i enhances and accelerates regression of inflammation, treatment starts immediately after mice have been placed back on normal diet (ND) and continue treatment for 4 weeks during recovery period, and mouse livers are collected weekly during this period, i.e., 4 data points to measure rate of recovery. The extent of recovery is quantified by NAFLD score and changes in immune cell infiltration are quantified by H&E. CD45+, CD68+ cells as described elsewhere herein, and stain specifically for CD3+, CD4+, CD8+ cells in selected set of samples.

Fibrosis. Because the factors involved in the natural history of NAFLD are not well defined, the relative contributions of steatosis, inflammation, associated oxidative stress, and other metabolic changes to the development and progression of remain unclear. However, the convergence of most or all of these appear to be required. The present data demonstrate that Sigma1i can prevent steatosis and that it can also mitigate the progression of steatosis. Thus, it is tested whether Sigma1i with CT189 can prevent fibrosis if mice are treated after the onset of steatosis but prior to the onset of fibrosis; in this case, treatment is started at week 8 and daily dosing continued until week 12, or until vehicle treated mice develop fibrosis. It is also tested in mice that have already developed fibrosis whether CT189 eliminates or prevents the progression of fibrotic lesions; in this case, treatment is started ˜week 12 to week 16 or until vehicle treated mice develop the equivalent of stage 4 fibrosis. Finally, drug assisted recovery if HFD is stopped and mice are returned to ND at 12 (NASH with fibrosis) is evaluated. Fibrotic lesions are detected and measured with pico-Sirius Red, molecular analysis of fibrogenic gene expression.

Blood-based biomarkers of liver response to Sigma1i targeted pharmacotherapy: Serum levels of ALT and AST. Serum levels of ALT and AST are monitored in response to Sigma1i treatment and it is determined whether changes in serum levels of ALT and AST track with/correspond with histological improvements. Serum ALT and AST levels from tail vein blood draws are measured using the materials and methods described elsewhere herein.

Changes in biomarkers of metabolic comorbidities in response to Sigma1i targeted pharmacotherapy: Serum levels of insulin, glucose, triglycerides (TGs), cholesterol. During each of the treatment timeframes described elsewhere herein, serum levels of glucose, insulin, triglycerides, and cholesterol in response to Sigma1i treatment are monitored, using insulin kit, glucose detector, total TG and cholesterol kits or by LC-MS/MS.

Hepatocellular carcinoma. The present data show that Sigma1i has anti-tumor properties, and HCC cells are responsive/sensitive to Sigma1i (Kim et al 2012 (PMID: 22925888); Schrock et al 2013 (PMID: 24006496); Kim and Maher 2017 (PMID: 28744586); Oyer et al 2019 (PMID: 31695608)). Therefore, it is tested whether Sigma1i with CT189 can prevent HCC if mice are treated after the onset of steatosis but prior to the emergence of HCC lesions. In this case, one starts treatment at week 20 and continues daily dosing until week 24, or until vehicle treated mice develop HCC lesions. It is also tested with mice that have already developed HCC lesions whether CT189 eliminates or prevents progression of these neoplastic lesions. In this case, treatment is started ˜week 24 to week 28 or until vehicle treated mice develop the equivalent of end stage tumor burden. The effect of continued HFD is evaluated by starting treatment after mice are returned to ND at week 20 (preventative protocol) and week 24 (for curative protocol).

### Example 7: Materials and Methods

Using the complete SIGMAR1 knockout (KO) C57BL/6 compared to WT SIGMAR1 C57BL/6 mice, one can evaluate the requirement for SIGMAR1/Sigma1 in the progression from healthy liver through NAFLD stages: hepatic steatosis (8 weeks), NASH with inflammatory fibrosis development (12 weeks) with high fat western diet (HFWD, WD containing 21.1% fat, 41% Sucrose, and 1.25% Cholesterol by weight (Teklad diets, TD.120528) and a high sugar solution (23.1 g/L D-fructose (Sigma-Aldrich, F0127) and 18.9 g/L D-glucose (Sigma-Aldrich, G8270)) alone compared to diet of HFWD+CCl₄ (administered weekly), which accelerates and amplifies HFWD induced NASH with associated fibrosis, progression to cirrhosis (24 weeks) and increase the frequency, number, and size of HCC lesions (24 weeks). All arms are compared to normal diet normal chow diet (ND, Lab diet, Rodent diet 20, #5053).

Histological markers of NAFL disease progression in the absence of Sigma1. Formalin-fixed, paraffin-embedded liver sections are stained with hematoxylin and eosin (H&E) for tissue architecture and assessment of liver histology, Sirius Red stain (Sigma, 365548-5G)/Fast Green (Sigma, F258) of collagen to assess fibrosis, oil-red-O stain for neutral lipid accumulation in the liver, and periodic acid-Schiff (PAS) to assess glycogen accumulation. NAFLD activity score and fibrosis stage are measured by an experienced pathologist blinded to treatment conditions, using the NASH CRN scoring system.

Molecular markers of fibrosis. Changes in expression of fibrogenic genes Collagen-alpha1, beta-PDGFR, TGF-beta, and Timp-1 are evaluated by qRT-PCR. Additionally, hepatic stellate cell (HSC) activation is evaluated by immunostaining for desmin (identify HSCs) and alpha-SMA (detect activated HSCs). Hepatocyte apoptosis is monitored by cleaved Caspase and/or TUNEL staining.

Inflammation markers. Inflammation is detected by immunostaining for CD45 (pan-leukocyte antigen) and CD68 (monocyte/macrophage infiltration). For CD45 positive sections, increased infiltration of CD3⁺, CD4⁺, and CD8⁺ positive immune cells, which have been linked to experimental NASH models including the HFWD/CCl₄ diet model, are monitored.

The present data indicate that Sigma1 modulation can suppress proinflammatory IL-17, IL-6, and TNFα (FIG. 7). Soluble IL-17 (sIL-17) is monitored as intrahepatic CD4+T lymphocytes produce elevated levels of IL-17 in NAFLD. IL-6 and TNFα are also monitored, as dietary and genetic obesity promote liver inflammation and tumorigenesis by enhancing IL-6 and TNFα expression.

Liver enzymes. Elevated serum ALT (alanine transaminase) and AST (aspartate transaminase) are biomarkers of NAFLD progression/liver dysfunction. Serum ALT and AST activity are monitored with fluorometric kits (Biovision) and/or measured using VITROS 5.1 FS (Ortho Clinical Diagnostics). Total serum triglyceride (TG) and cholesterol levels are measured using commercially available testing kits and/or by LC-Mass Spectrometry, and/or using the VITROS 5.1 FS (Ortho Clinical Diagnostics).

Insulin resistance and glucose tolerance. Insulin resistance and glucose tolerance are symptoms/indicators of metabolic syndrome and NAFLD. Metabolic syndrome associated biomarkers are monitored. Fasting and non-fasting plasma insulin levels are tested with the Ultrasensitive Mouse Insulin ELISA kit (Crystal Chem, 90080). Fasting and non-fasting blood glucose levels are monitored with the One Touch Ultra (Life Scan). In animals receiving CCl₄, blood is analyzed seven days after the most recent administration. For assessment for insulin resistance in the non-fasting, homeostatic model, QUICKI is calculated as described elsewhere (Lee, et al., 2008, Amer. J. Physiol Endocrinol. Metab. 294, E261-270; Bowe, et al., 2014, J. Endocrinol. 222:G13-25).

SIGMAR1/Sigma1 status. In WT C57BL/6 mice the status of SIGMAR1 mRNA transcripts are monitored and evaluated by qRT-PCR, and Sigma1 protein levels and distribution patterns are monitored and evaluated by immunohistochemical (IHC) staining in liver tissue at key stages of disease progression: steatosis (8 weeks), steatohepatitis with inflammation and fibrosis (12 weeks), and HCC lesions (24 weeks).

The disclosures of each and every patent, patent application, and publication cited herein are hereby incorporated herein by reference in their entirety.

While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. (canceled)

2. A method of treating, ameliorating, or preventing a disease or condition in a subject, the method comprising administering to the subject an effective amount of an agent that reduces or prevents SIGMAR1 transcription, or reduces or inhibits Sigma1 activity or expression, in the subject,
wherein the disease or condition is at least one selected from the group consisting of:
hepatic steatosis (fatty liver) or an inflammation associated with steatohepatitis;
nonalcoholic fatty liver disease (NAFLD); or
nonalcoholic steatohepatitis (NASH).

3-4. (canceled)

5. The method of claim 2, wherein at least one of the following applies:
(a) the disease or condition is NAFLD, and the method reverses, slows, or prevents progression of NAFLD to NASH in the subject;
(b) the disease or condition is NASH, and the method reverses, slows, or prevents progression of NASH to hepatocellular carcinoma (HCC) in the subject.

6-13. (canceled)

14. The method of claim 2, wherein the administering reverses, prevents, or ameliorates at least one of the following: steatosis, inflammation, hepatocyte ballooning, fibrosis, hepatic stellate cell (HSC) activation, serum liver enzyme increase, insulin resistance/glucose tolerance, and HCC lesion formation.

15. The method of claim 2, wherein at least one of the following applies:
(a) the agent comprises a CRISPR system against SIGMAR1 or a nucleic acid against SIGMAR1 selected from a siRNA, a shRNA, a microRNA, or antisense polynucleotide;
(b) the agent comprises a compound of Formula (I) or a salt, solvate, or N-oxide thereof, or any combinations thereof: 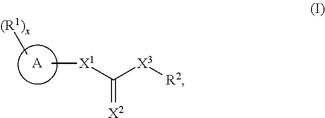
wherein:
ring A is a monocyclic or bicyclic aryl or a monocyclic or bicyclic heteroaryl ring, and wherein the aryl or heteroaryl ring is optionally substituted with 0-4 R¹ groups;
each occurrence of R¹ is independently selected from the group consisting of —C₁-C₆ alkyl, —C₁-C₆ fluoroalkyl, —C₁-C₆ heteroalkyl, F, Cl, Br, I, —CN, —NO₂, —OR³, —SR³, —S(═O)R³, —S(═O)₂R³, —NHS(═O)₂R³, —C(═O)R³, —OC(═O)R³, —CO₂R³, —OCO₂R³, —CH(R³)₂, —N(R³)₂, —C(═O)N(R³)₂, —OC(═O)N(R³)₂, —NHC(═O)NH(R³), —NHC(═O)R³, —NHC(═O)OR³, —C(OH)(R³)₂, and —C(NH₂)(R³)₂;
each occurrence of R² is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl or cycloalkyl group is optionally substituted with 0-5 R¹ groups, or X³ and R² combine to form a (C₃-C₇)heterocycloalkyl group, optionally substituted with 0-2 R¹ groups;
each occurrence of R³ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted with 0-5 R¹ groups;
X¹ is —CH₂—, —S—, —O— or —(NR₂)—;
X² is ═CH₂, ═S, —O or ═NR₂; and
X³ is —S—, —O—, or —NR₂—;
(c) the agent comprises a compound of Formula (II) or a salt, solvate, or N-oxide thereof, or any combinations thereof:
R^{A}—R^{B} (II),
wherein:
R^{A} is selected from the group consisting of 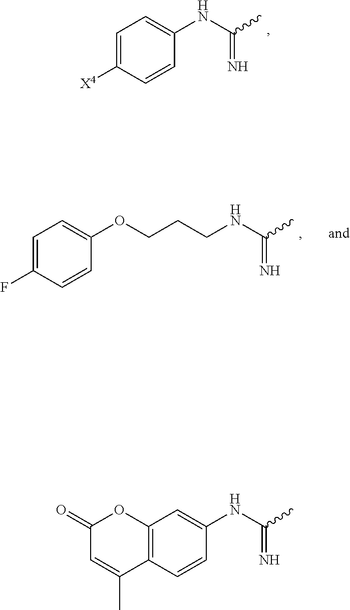
X⁴ is selected from the group consisting of methoxy, F, Cl, Br, and I; and
R^{B} is selected from the group consisting of: 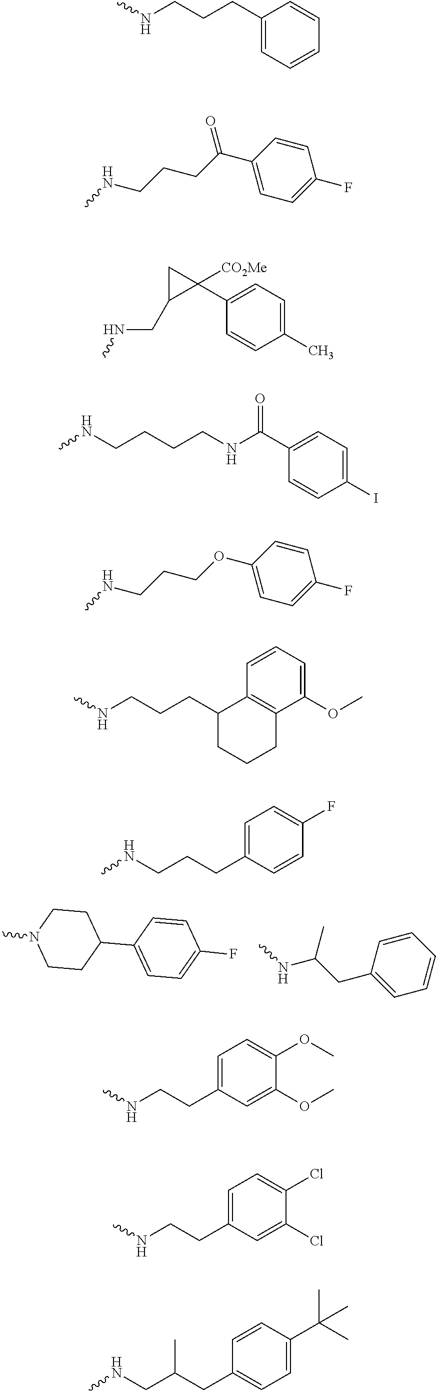 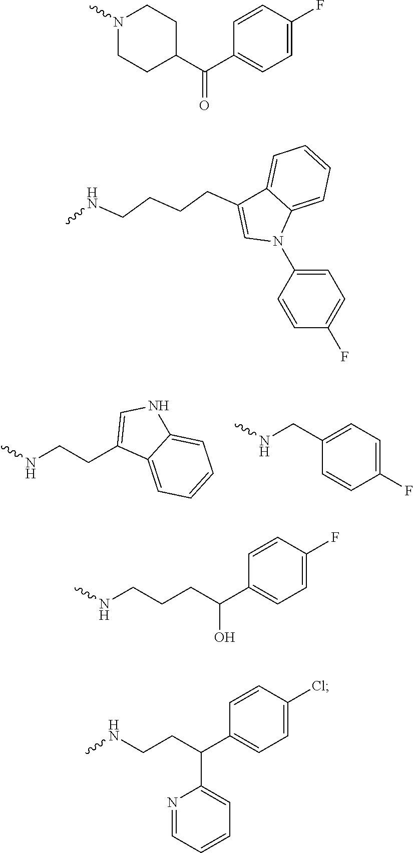
(d) the agent comprises a compound of Formula (III) or a salt, solvate, or N-oxide thereof, or any combinations thereof: 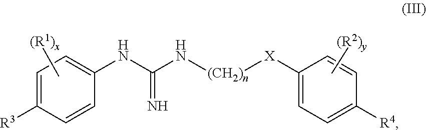
wherein:
each occurrence of R¹ and R² is independently selected from the group consisting of —C₁-C₆ alkyl, —C₁-C₆ fluoroalkyl, —C₁-C₆ heteroalkyl, F, Cl, Br, I, —CN, —NO₂, —OR⁵, —SR⁵, —S(═O)R⁵, —S(═O)₂R⁵, —NHS(═O)₂R⁵, —C(═O)R⁵, —OC(═O)R⁵, —CO₂R⁵—OCO₂R⁵, —CH(R⁵)₂, —N(R⁵)₂, —C(═O)N(R⁵)₂, —OC(═O)N(R⁵)₂, —NHC(═O)NH(R⁵), —NHC(═O)R⁵, —NHC(═O)OR⁵, —C(OH)(R⁵)₂, and —C(NH₂)(R⁵)₂;
R³ is selected from the group consisting of —C₁-C₆ alkyl, —C₁-C₆ fluoroalkyl, —C₁-C₆ alkoxy, F, Cl, Br, and I;
R⁴ is selected from the group consisting of —C₁-C₆ alkyl, —C₁-C₆ alkoxy, F, Cl, Br, and I;
each occurrence of R⁵ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted;
X is selected from the group consisting of CH₂, C—O, or O;
n is 1, 2, or 3;
x is 0, 1, 2, 3, or 4; and
y is 0, 1, 2, 3, or 4;
(e) the agent comprises a compound selected from the group consisting of: 1-(3-(4-fluorophenoxy)propyl)-3-(4-iodophenyl)guanidine (Compound A); 1-(3-(4-fluorophenoxy)propyl)-3-(4-methoxyphenyl)guanidine (Compound B); 1-(n-propyl)-3-(4-iodophenyl)guanidine (Compound C); 1-(n-propyl)-3-(4-methoxyphenyl)guanidine (Compound D); 1-(3-(4-fluorophenoxy)propyl)-3-(4-trifluoromethylphenyl)guanidine (Compound F); 1-(3-(4-fluorophenoxy)propyl)-3-(4-chlorophenyl)guanidine (Compound G); or a salt, solvate or N-oxide thereof, and any combinations thereof;
(f) the agent comprises a compound selected from the group consisting of: 1,3-bis(3-(4-fluorophenoxy)propyl)guanidine (Compound E); 1-(3-(4-fluorophenoxy)propyl)-3-(4-methyl-2-oxo-2H-chromen-7-yl)guanidine) (Compound H); or a salt, solvate or N-oxide thereof, and any combinations thereof.

16. The method of claim 15, wherein the subject is administered a viral vector expressing the nucleic acid.

17. The method of claim 16, wherein the nucleic acid is expressed in the liver of the subject.

18-20. (canceled)

21. The method of claim 15, wherein the agent comprises at least one selected from (b)-(f), and wherein at least one of the following applies:
(a) the compound is administered as a pharmaceutical composition further comprising a pharmaceutically acceptable carrier,
(b) the subject is further administered at least one additional agent that treats, prevents, or ameliorates one of NAFDL, NASH, or HCC,
(c) the compound is administered by a route comprising oral, nasal, rectal, intravaginal, parenteral, buccal, sublingual or topical,
(d) the subject is a mammal,
(e) the subject is a human.

22-25. (canceled)

26. A method of determining or evaluating risk that a subject suffering from NAFLD will progress to NASH, or risk that a subject suffering from NASH will progress to HCC,
the method comprising measuring levels of SIGMAR1 transcription or Sigma1 activity/expression in the subject, and comparing the measured levels to those levels of SIGMAR1 transcription or Sigma1 activity/expression in a control sample.

27. The method of claim 26, wherein at least one of the following applies:
(a) the subject at risk of progressing to NASH is counseled to receive an effective amount of an agent that reduces or prevents SIGMAR1 transcription, or reduces or inhibits Sigma1 activity or expression;
(b) the subject at risk of progressing to NASH is administered an effective amount of an agent that reduces or prevents SIGMAR1 transcription, or reduces or inhibits Sigma1 activity or expression;
(c) the subject at risk of progressing to HCC is counseled to receive an effective amount of an agent that modulates SIGMAR1 transcription, or modulates Sigma1 activity or expression;
(d) the subject at risk of progressing to HCC is counseled to receive an effective amount of an agent that modulates SIGMAR1 transcription, or modulates Sigma1 activity or expression
(e) the control sample is from at least of the following:
a subject suffering from NAFLD;
a subject not suffering from NAFLD;
a subject suffering from NASH;
a subject not suffering from NASH;
a subject suffering from HCC;
a subject not suffering from HCC;
a subject suffering from NAFLD who has not progressed to NASH or is known not to be at risk of progressing to NASH;
a subject suffering from NAFLD who has progressed to NASH or is known to be at risk of progressing to NASH;
a subject suffering from NASH who has not progressed to HCC or is known not to be at risk of progressing to HCC;
a subject suffering from NASH who has progressed to HCC or is known to be at risk of progressing to HCC.

28. The method of claim 26, wherein at least one of the following applies:
(a) the agent comprises a CRISPR system against SIGMAR1 or a nucleic acid against SIGMAR1 selected from a siRNA, a shRNA, a microRNA, or antisense polynucleotide;
(b) the agent comprises a compound of Formula (I) or a salt, solvate, or N-oxide thereof, or any combinations thereof: 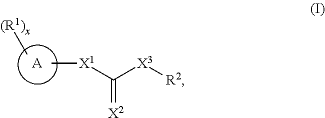
wherein:
ring A is a monocyclic or bicyclic aryl or a monocyclic or bicyclic heteroaryl ring, and wherein the aryl or heteroaryl ring is optionally substituted with 0-4 R¹ groups;
each occurrence of R¹ is independently selected from the group consisting of —C₁-C₆ alkyl, —C₁-C₆ fluoroalkyl, —C₁-C₆ heteroalkyl, F, Cl, Br, I, —CN, —NO₂, —OR³, —SR³, —S(═O)R³, —S(═O)₂R³, —NHS(═O)₂R³, —C(═O)R³, —OC(═O)R³, —CO₂R³, —OCO₂R³, —CH(R³)₂, —N(R³)₂, —C(═O)N(R³)₂, —OC(═O)N(R³)₂, —NHC(═O)NH(R³), —NHC(═O)R³, —NHC(═O)OR³, —C(OH)(R³)₂, and —C(NH₂)(R³)₂;
each occurrence of R² is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl or cycloalkyl group is optionally substituted with 0-5 R¹ groups, or X³ and R² combine to form a (C₃-C₇)heterocycloalkyl group, optionally substituted with 0-2 R¹ groups;
each occurrence of R³ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted with 0-5 R¹ groups;
X¹ is —CH₂—, —S—, —O— or —(NR₂)—;
X² is ═CH₂, ═S, —O or ═NR₂; and
X³ is —S—, —O—, or —NR₂—;
(c) the agent comprises a compound of Formula (II) or a salt, solvate, or N-oxide thereof, or any combinations thereof:
R^{A}—R^{B} (II),
wherein:
R^{A} is selected from the group consisting of 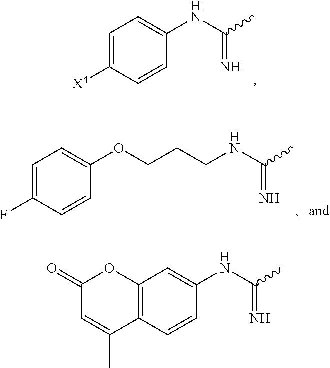
X⁴ is selected from the group consisting of methoxy, F, Cl, Br, and I; and
R^{B} is selected from the group consisting of: 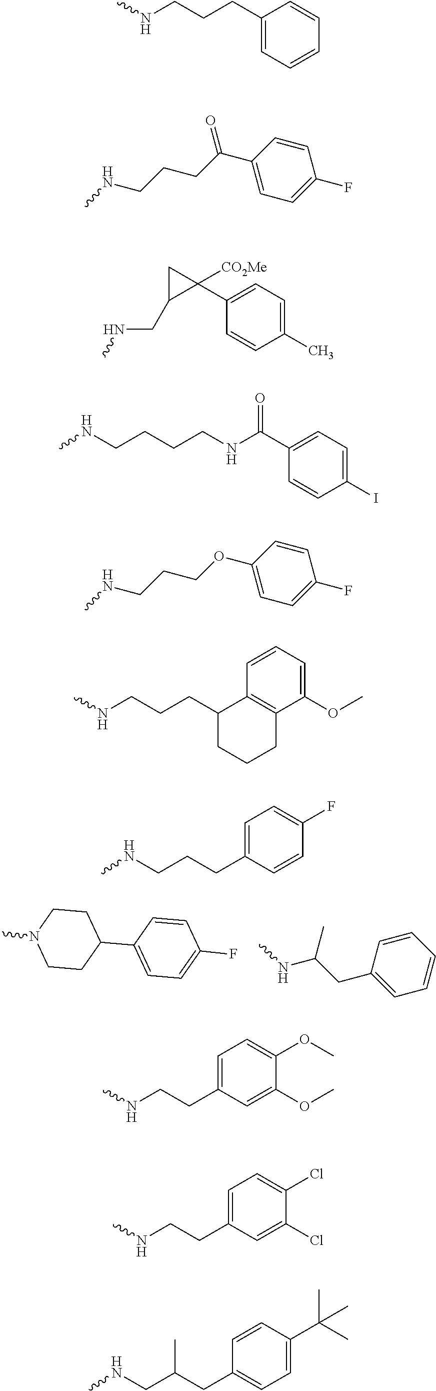 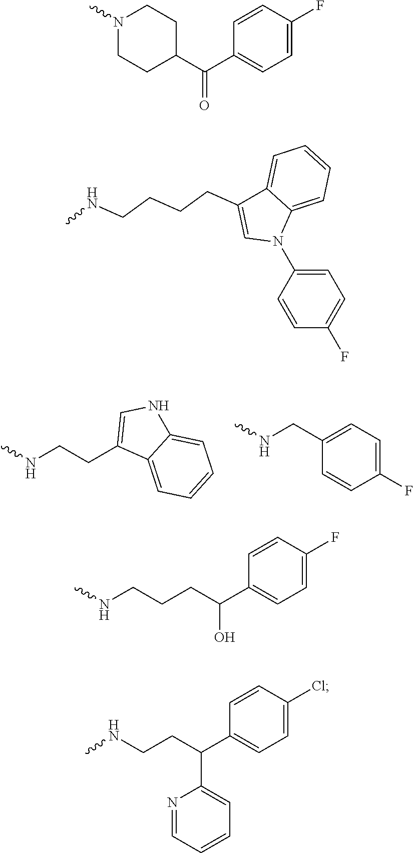
(d) the agent comprises a compound of Formula (III) or a salt, solvate, or N-oxide thereof, or any combinations thereof: 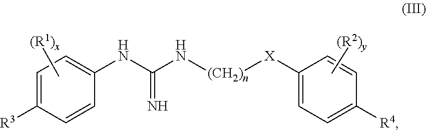
wherein:
each occurrence of R¹ and R² is independently selected from the group consisting of —C₁-C₆ alkyl, —C₁-C₆ fluoroalkyl, —C₁-C₆ heteroalkyl, F, Cl, Br, I, —CN, —NO₂, —OR⁵, —SR⁵, —S(═O)R⁵, —S(═O)₂R⁵, —NHS(═O)₂R⁵, —C(═O)R⁵, —OC(═O)R⁵, —CO₂R⁵, —OCO₂R⁵, —CH(R⁵)₂, —N(R⁵)₂, —C(═O)N(R⁵)₂, —OC(═O)N(R⁵)₂, —NHC(═O)NH(R⁵), —NHC(═O)R⁵, —NHC(═O)OR⁵, —C(OH)(R⁵)₂, and —C(NH₂)(R⁵)₂;
R³ is selected from the group consisting of —C₁-C₆ alkyl, —C₁-C₆ fluoroalkyl, —C₁-C₆ alkoxy, F, Cl, Br, and I;
R⁴ is selected from the group consisting of —C₁-C₆ alkyl, —C₁-C₆ alkoxy, F, Cl, Br, and I;
each occurrence of R⁵ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted;
X is selected from the group consisting of CH₂, C═O, or O;
n is 1, 2, or 3;
x is 0, 1, 2, 3, or 4; and
y is 0, 1, 2, 3, or 4;
(e) the agent comprises a compound selected from the group consisting of: 1-(3-(4-fluorophenoxy)propyl)-3-(4-iodophenyl)guanidine (Compound A); 1-(3-(4-fluorophenoxy)propyl)-3-(4-methoxyphenyl)guanidine (Compound B); 1-(n-propyl)-3-(4-iodophenyl)guanidine (Compound C); 1-(n-propyl)-3-(4-methoxyphenyl)guanidine (Compound D); 1-(3-(4-fluorophenoxy)propyl)-3-(4-trifluoromethylphenyl)guanidine (Compound F); 1-(3-(4-fluorophenoxy)propyl)-3-(4-chlorophenyl)guanidine (Compound G); or a salt, solvate or N-oxide thereof, and any combinations thereof;
(f) the agent comprises a compound selected from the group consisting of: 1,3-bis(3-(4-fluorophenoxy)propyl)guanidine (Compound E); 1-(3-(4-fluorophenoxy)propyl)-3-(4-methyl-2-oxo-2H-chromen-7-yl)guanidine) (Compound H); or a salt, solvate or N-oxide thereof, and any combinations thereof.

29. The method of claim 28, wherein the subject is administered a viral vector expressing the nucleic acid.

30. The method of claim 29, wherein the nucleic acid is expressed in the liver of the subject.

31. The method of claim 28, wherein the agent comprises at least one selected from (b)-(f), and wherein at least one of the following applies:
(a) the compound is administered as a pharmaceutical composition further comprising a pharmaceutically acceptable carrier,
(b) the subject is further administered at least one additional agent that treats, prevents, or ameliorates one of NAFDL, NASH, or HCC,
(c) the compound is administered by a route comprising oral, nasal, rectal, intravaginal, parenteral, buccal, sublingual or topical,
(d) the subject is a mammal,
(e) the subject is a human.

32. A method of treating, ameliorating, or preventing accumulation of lipid droplets (LDs) in a cell, the method comprising contacting the cell with an agent that reduces or prevents SIGMAR1 transcription, or reduces or inhibits Sigma1 activity or expression, in the cell.

33. The method of claim 32, wherein at least one of the following applies:
(a) the agent comprises a CRISPR system against SIGMAR1 or a nucleic acid against SIGMAR1 selected from a siRNA, a shRNA, a microRNA, or antisense polynucleotide;
(b) the agent comprises a compound of Formula (I) or a salt, solvate, or N-oxide thereof, or any combinations thereof: 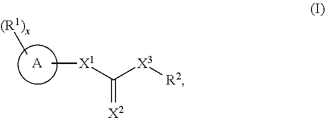
wherein:
ring A is a monocyclic or bicyclic aryl or a monocyclic or bicyclic heteroaryl ring, and wherein the aryl or heteroaryl ring is optionally substituted with 0-4 R¹ groups;
each occurrence of R¹ is independently selected from the group consisting of —C₁-C₆ alkyl, —C₁-C₆ fluoroalkyl, —C₁-C₆ heteroalkyl, F, Cl, Br, I, —CN, —NO₂, —OR³, —SR³, —S(═O)R³, —S(═O)₂R³, —NHS(═O)₂R³, —C(═O)R³, —OC(═O)R³, —CO₂R³, —OCO₂R³, —CH(R³)₂, —N(R³)₂, —C(═O)N(R³)₂, —OC(═O)N(R³)₂, —NHC(═O)NH(R³), —NHC(═O)R³, —NHC(═O)OR³, —C(OH)(R³)₂, and —C(NH₂)(R³)₂;
each occurrence of R² is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl or cycloalkyl group is optionally substituted with 0-5 R¹ groups, or X³ and R² combine to form a (C₃-C₇)heterocycloalkyl group, optionally substituted with 0-2 R¹ groups;
each occurrence of R³ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted with 0-5 R¹ groups;
X¹ is —CH₂—, —S—, —O— or —(NR₂)—;
X² is ═CH₂, ═S, ═O or ═NR₂; and
X³ is —S—, —O—, or —NR₂—;
(c) the agent comprises a compound of Formula (II) or a salt, solvate, or N-oxide thereof, or any combinations thereof:
R^{A}—R^{B} (II),
wherein:
R^{A} is selected from the group consisting of 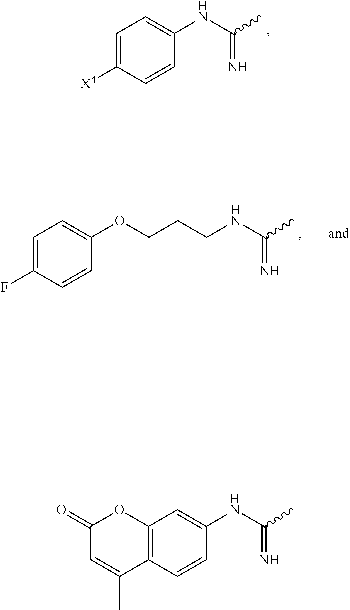
X⁴ is selected from the group consisting of methoxy, F, Cl, Br, and I; and
R^{B} is selected from the group consisting of: 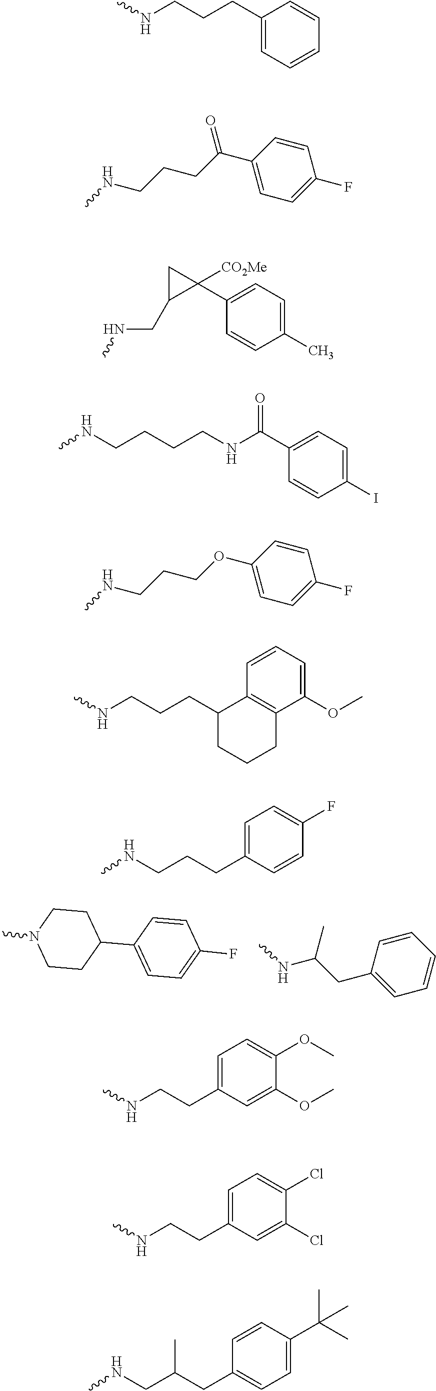 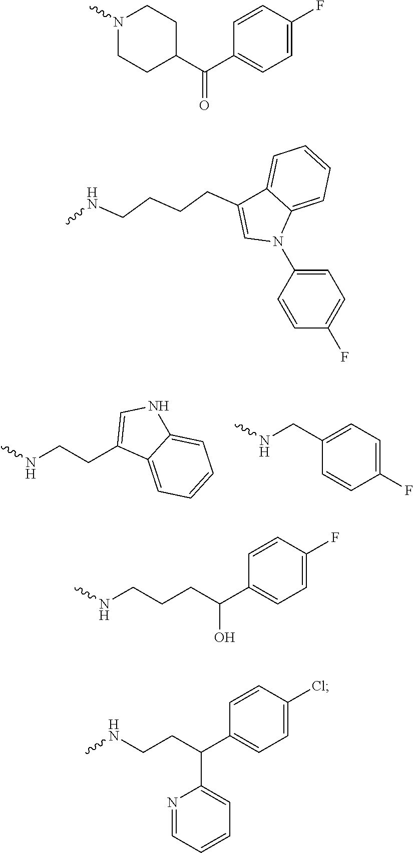
(d) the agent comprises a compound of Formula (III) or a salt, solvate, or N-oxide thereof, or any combinations thereof: 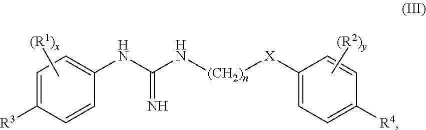
wherein:
each occurrence of R¹ and R² is independently selected from the group consisting of —C₁-C₆ alkyl, —C₁-C₆ fluoroalkyl, —C₁-C₆ heteroalkyl, F, Cl, Br, I, —CN, —NO₂, —OR⁵, —SR⁵, —S(═O)R⁵, —S(═O)₂R⁵, —NHS(═O)₂R⁵, —C(═O)R⁵, —OC(═O)R⁵, —CO₂R⁵, —OCO₂R⁵, —CH(R⁵)₂, —N(R⁵)₂, —C(═O)N(R⁵)₂, —OC(═O)N(R⁵)₂, —NHC(═O)NH(R⁵), —NHC(═O)R⁵, —NHC(═O)OR⁵, —C(OH)(R⁵)₂, and —C(NH₂)(R⁵)₂;
R³ is selected from the group consisting of —C₁-C₆ alkyl, —C₁-C₆ fluoroalkyl, —C₁-C₆ alkoxy, F, Cl, Br, and I;
R⁴ is selected from the group consisting of —C₁-C₆ alkyl, —C₁-C₆ alkoxy, F, Cl, Br, and I;
each occurrence of R⁵ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted;
X is selected from the group consisting of CH₂, C═O, or O;
n is 1, 2, or 3;
x is 0, 1, 2, 3, or 4; and
y is 0, 1, 2, 3, or 4;
(e) the agent comprises a compound selected from the group consisting of: 1-(3-(4-fluorophenoxy)propyl)-3-(4-iodophenyl)guanidine (Compound A); 1-(3-(4-fluorophenoxy)propyl)-3-(4-methoxyphenyl)guanidine (Compound B); 1-(n-propyl)-3-(4-iodophenyl)guanidine (Compound C); 1-(n-propyl)-3-(4-methoxyphenyl)guanidine (Compound D); 1-(3-(4-fluorophenoxy)propyl)-3-(4-trifluoromethylphenyl)guanidine (Compound F); 1-(3-(4-fluorophenoxy)propyl)-3-(4-chlorophenyl)guanidine (Compound G); or a salt, solvate or N-oxide thereof, and any combinations thereof;
(f) the agent comprises a compound selected from the group consisting of: 1,3-bis(3-(4-fluorophenoxy)propyl)guanidine (Compound E); 1-(3-(4-fluorophenoxy)propyl)-3-(4-methyl-2-oxo-2H-chromen-7-yl)guanidine) (Compound H); or a salt, solvate or N-oxide thereof, and any combinations thereof.
